SEQUENCE LISTING (47 sequences)

SEQ ID NO: 1 - protein, 1210 aa, Homo sapiens [sequence omitted]

SEQ ID NO: 2 - protein, 171 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQ

SEQ ID NO: 3 - protein, 141 aa, Homo sapiens
VCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 4 - protein, 14 aa, synthetic construct
  REGION 1..14: note = Description of Artificial Sequence: Synthetic peptide
VTGSGWGPEPGGGS

SEQ ID NO: 5 - protein, 23 aa, Homo sapiens
IATGMVGALLLLLVVALGIGLFM

SEQ ID NO: 6 - protein, 6 aa, synthetic construct
  REGION 1..6: note = Description of Artificial Sequence: Synthetic peptide
KSGSGS

SEQ ID NO: 7 - protein, 4 aa, synthetic construct
  REGION 1..4: note = Description of Artificial Sequence: Synthetic peptide
KRSD

SEQ ID NO: 8 - protein, 5 aa, synthetic construct
  REGION 1..5: note = Description of Artificial Sequence: Synthetic peptide
KRSDK

SEQ ID NO: 9 - protein, 5 aa, synthetic construct
  REGION 1..5: note = Description of Artificial Sequence: Synthetic peptide
SGGGG

SEQ ID NO: 10 - protein, 5 aa, synthetic construct
  REGION 1..5: note = Description of Artificial Sequence: Synthetic peptide
SGAGG

SEQ ID NO: 11 - protein, 5 aa, synthetic construct
  REGION 1..5: note = Description of Artificial Sequence: Synthetic peptide
KRADK

SEQ ID NO: 12 - protein, 14 aa, synthetic construct
  REGION 1..14: note = Description of Artificial Sequence: Synthetic peptide
RRRSGGGGSGGGGS

SEQ ID NO: 13 - protein, 11 aa, synthetic construct
  REGION 1..11: note = Description of Artificial Sequence: Synthetic peptide
SGGGGSGGGGS

SEQ ID NO: 14 - protein, 5 aa, synthetic construct
  REGION 1..5: note = Description of Artificial Sequence: Synthetic peptide
  REGION 1..5: note = See specification as filed for detailed description of substitutions and preferred embodiments
GGGGS

SEQ ID NO: 15 - protein, 35 aa, Homo sapiens
RRRHIVRKRTLRRLLQERELVEPLTPSGEAPNQAL

SEQ ID NO: 16 - protein, 9 aa, synthetic construct
  REGION 1..9: note = Description of Artificial Sequence: Synthetic peptide
RRRHIVRKR

SEQ ID NO: 17 - protein, 8 aa, synthetic construct
  REGION 1..8: note = Description of Artificial Sequence: Synthetic peptide
RRRHIVRK

SEQ ID NO: 18 - protein, 7 aa, synthetic construct
  REGION 1..7: note = Description of Artificial Sequence: Synthetic peptide
RRRHIVR

SEQ ID NO: 19 - protein, 6 aa, synthetic construct
  REGION 1..6: note = Description of Artificial Sequence: Synthetic peptide
RRRHIV

SEQ ID NO: 20 - protein, 5 aa, synthetic construct
  REGION 1..5: note = Description of Artificial Sequence: Synthetic peptide
RRRHI

SEQ ID NO: 21 - protein, 4 aa, synthetic construct
  REGION 1..4: note = Description of Artificial Sequence: Synthetic peptide
RRRH

SEQ ID NO: 22 - protein, 24 aa, Homo sapiens
MRPSGTAGAALLALLAALCPASRA

SEQ ID NO: 23 - protein, 22 aa, Homo sapiens
MLLLVTSLLLCELPHPAFLLIP

SEQ ID NO: 24 - protein, 20 aa, Homo sapiens
MVLQTQVFISLLLWISGAYG

SEQ ID NO: 25 - protein, 16 aa, Homo sapiens
MPLLLLLPLLWAGALA

SEQ ID NO: 26 - protein, 335 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 27 - protein, 219 aa, synthetic construct
  REGION 1..219: note = Description of Artificial Sequence: Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGPSIATGMVGALLLLLVVALGIGLFM

SEQ ID NO: 28 - protein, 209 aa, synthetic construct
  REGION 1..209: note = Description of Artificial Sequence: Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTGSGWGPEPGGGSPSIATGMVGALLLLLVVALGIGLFM

SEQ ID NO: 29 - protein, 360 aa, synthetic construct
  REGION 1..360: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRR

SEQ ID NO: 30 - protein, 366 aa, synthetic construct
  REGION 1..366: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKR

SEQ ID NO: 31 - protein, 371 aa, synthetic construct
  REGION 1..371: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRSGGGGSGGGGS

SEQ ID NO: 32 - protein, 368 aa, synthetic construct
  REGION 1..368: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMSGGGGSGGGGS

SEQ ID NO: 33 - protein, 244 aa, synthetic construct
  REGION 1..244: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGPSIATGMVGALLLLLVVALGIGLFMRRR

SEQ ID NO: 34 - protein, 250 aa, synthetic construct
  REGION 1..250: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGPSIATGMVGALLLLLVVALGIGLFMRRRHIVRKR

SEQ ID NO: 35 - protein, 255 aa, synthetic construct
  REGION 1..255: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGPSIATGMVGALLLLLVVALGIGLFMRRRSGGGGSGGGGS

SEQ ID NO: 36 - protein, 252 aa, synthetic construct
  REGION 1..252: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGPSIATGMVGALLLLLVVALGIGLFMSGGGGSGGGGS

SEQ ID NO: 37 - protein, 234 aa, synthetic construct
  REGION 1..234: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTGSGWGPEPGGGSPSIATGMVGALLLLLVVALGIGLFMRRR

SEQ ID NO: 38 - protein, 240 aa, synthetic construct
  REGION 1..240: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTGSGWGPEPGGGSPSIATGMVGALLLLLVVALGIGLFMRRRHIVRKR

SEQ ID NO: 39 - protein, 245 aa, synthetic construct
  REGION 1..245: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTGSGWGPEPGGGSPSIATGMVGALLLLLVVALGIGLFMRRRSGGGGSGGGGS

SEQ ID NO: 40 - protein, 242 aa, synthetic construct
  REGION 1..242: note = Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQTGSGWGPEPGGGSPSIATGMVGALLLLLVVALGIGLFMSGGGGSGGGGS

SEQ ID NO: 41 - protein, 43 aa, synthetic construct
  REGION 1..43: note = Description of Artificial Sequence: Synthetic polypeptide
DEARKAIARVKRESKRIVEDAERLIREAAAASEKISREAERLI

SEQ ID NO: 42 - protein, 10 aa, synthetic construct
  REGION 1..10: note = Description of Artificial Sequence: Synthetic peptide
GGGGSGGGGS

SEQ ID NO: 43 - protein, 58 aa, Homo sapiens
IATGMVGALLLLLVVALGIGLFMRRRHIVRKRTLRRLLQERELVEPLTPSGEAPNQAL

SEQ ID NO: 44 - protein, 26 aa, synthetic construct
  REGION 1..26: note = Description of Artificial Sequence: Synthetic peptide
IATGMVGALLLLLVVALGIGLFMRRR

SEQ ID NO: 45 - protein, 32 aa, synthetic construct
  REGION 1..32: note = Description of Artificial Sequence: Synthetic polypeptide
IATGMVGALLLLLVVALGIGLFMRRRHIVRKR

SEQ ID NO: 46 - protein, 37 aa, synthetic construct
  REGION 1..37: note = Description of Artificial Sequence: Synthetic polypeptide
IATGMVGALLLLLVVALGIGLFMRRRSGGGGSGGGGS

SEQ ID NO: 47 - protein, 34 aa, synthetic construct
  REGION 1..34: note = Description of Artificial Sequence: Synthetic polypeptide
IATGMVGALLLLLVVALGIGLFMSGGGGSGGGGS

(19) United States
(12) Patent Application Publication
(10) Pub. No.: US 2024/0409610 A1
(43) Pub. Date: Dec. 12, 2024

(54) Novel Recombinant Cell Surface Markers

(71) Applicant: Lyell Immunopharma, Inc., South San Francisco, CA (US)

(72) Inventors: Howell Franklin Moffett, Seattle, WA (US); Marc Joseph Lajoie, Seattle, WA (US); Scott Edward Boyken, Seattle, WA (US)

(73) Assignee: Lyell Immunopharma, Inc., South San Francisco, CA (US)

(21) Appl. No.: 18/750,174

(22) Filed: Jun. 21, 2024

Related U.S. Application Data
(62) Division of application No. 17/207,523, filed on Mar. 19, 2021, now Pat. No. 12,054,531.
(60) Provisional application No. 63/137,022, filed on Jan. 13, 2021.
(60) Provisional application No. 62/992,806, filed on Mar. 20, 2020.

(51) Int. Cl. C07K 14/71; A61K 38/00; A61K 39/00; C07K 14/535; C07K 14/705; C07K 14/725; C07K 16/28; C12N 5/0783

(52) U.S. Cl. CPC C07K 14/71; A61K 39/4611; A61K 39/4631; A61K 39/464402; A61K 39/464404; A61K 39/464412; A61K 2239/28; A61K 2239/31; A61K 2239/38; C07K 14/535; C07K 14/7051; C07K 14/70596; C07K 16/2863; C12N 5/0636; A61K 38/00; C07K 2319/02; C07K 2319/03

(57) ABSTRACT

The present disclosure relates to EGFR-derived polypeptides containing short juxtamembrane sequences, nucleic acids encoding them, and methods of using them to improve cell surface expression of truncated EGFR markers.

### CROSS-REFERENCE TO RELATED APPLICATIONS

This application is a divisional of U.S. application Ser. No. 17/207,523, filed Mar. 19, 2021, which claims priority from U.S. Provisional Application 62/992,806, filed Mar. 20, 2020, and U.S. Provisional Application 63/137,022, filed Jan. 13, 2021. The disclosures of the aforementioned applications are incorporated herein by reference in their entirety.

### SEQUENCE LISTING

The instant application contains a Sequence Listing that has been submitted electronically in XML format. The Sequence Listing is hereby incorporated by reference in its entirety. The XML copy, created on Jun. 18, 2024, is named 026225_D1012_SL.xml and is 62,554 bytes in size.

### BACKGROUND OF THE INVENTION

The epidermal growth factor family of receptors tyrosine kinases (ErbBs) consists of four members: EGFR/ErbB1/HER1, ErbB2/HER2/Neu, ErbB3/HER3, and ErbB4/HER4 (Wieduwilt and Moasser, Cell Mol Life Sci. (2008) 65(10):1566-84). These receptors are widely expressed in epithelial, mesenchymal, and neuronal tissue and play critical roles in cell proliferation, differentiation, and development (Yano et al., Anticancer Res. (2003) 23(5A):3639-50). They are activated by ligands that induce either homo- or hetero-dimerization of the epidermal growth factor receptor (EGFR) homologs. EGFR is a 180 kDa monomeric glycoprotein comprising a large extracellular region, a single spanning transmembrane domain, an intracellular juxtamembrane region, a tyrosine kinase domain, and a C-terminal regulatory region. The extracellular region comprises four domains: Domains I and III are homologous ligand binding domains, and domains II and IV are cysteine rich domains (Ferguson, Annu Rev Biophys. (2008) 37:353-3).

The structured Domain III of human EGFR is targeted by the FDA licensed monoclonal antibody cetuximab (Erbitux®). Separating the cetuximab-binding ability of EGFR from its biological activity by selective truncation of the receptor offers the potential for an inert, fully human cell surface marker (Li et al., Cancer Cell (2005) 7(4):301-11; Wang et al., Blood (2011) 118(5):1255-63). However, a critical feature of a clinically useful cell surface marker is that the marker needs to be expressed at consistently high levels in the engineered cells such that the engineered cells can be sufficiently identified and targeted when needed.

### SUMMARY OF THE INVENTION

The present disclosure provides a recombinant polypeptide comprising an extracellular region, a transmembrane region, and an intracellular region, wherein the extracellular region comprises a human epidermal growth factor receptor (EGFR) Domain III sequence, and the intracellular region (i) comprises a juxtamembrane domain that is net-neutral or net-positively charged in the first at least three amino acids (ii) but lacks an active EGFR tyrosine kinase domain. In some embodiments, the polypeptide does not have any active tyrosine kinase domain.

In some embodiments, more than half of the amino acids of the juxtamembrane domain are glycine, serine, arginine, lysine, threonine, asparagine, glutamine, aspartic acid, glutamic acid, tyrosine, tryptophan, histidine, and/or proline. In some embodiments, the amino acid at each position of the juxtamembrane domain is selected according to Table 1. For example, the juxtamembrane domain comprises RRRHIVRKR (SEQ ID NO:16), RRRHIVRK (SEQ ID NO:17), RRRHIVR (SEQ ID NO:18), RRRHIV (SEQ ID NO:19), RRRHI (SEQ ID NO:20), RRRH (SEQ ID NO:21), RRR, RKR, or RR. In certain embodiments, the intracellular region does not contain any residue that is phosphorylated.

In some embodiments, the human EGFR Domain III sequence may comprise SEQ ID NO:2 or a functional variant thereof such as a sequence comprising at least 90% identity to SEQ ID NO:2. In some embodiments, the extracellular region further comprises, C-terminal to the Domain III sequence, (i) a sequence derived from EGFR Domain IV, (ii) an artificial sequence, or (iii) both (i) and (ii). In particular embodiments, the extracellular region comprises amino acids 334-504, 334-525, or 334-645 of SEQ ID NO:1.

In some embodiments, the transmembrane region is derived from a human EGFR transmembrane domain, optionally comprising SEQ ID NO:5.

In some embodiments, the recombinant polypeptide comprises a signal peptide derived from human EGFR, human granulocyte-macrophage colony-stimulating factor (GM-CSF), human Ig kappa, mouse Ig kappa, or human CD33. For example, the signal peptide may comprise SEQ ID NO:22, 23, 24, or 25.

In particular embodiments, the recombinant polypeptide comprises SEQ ID NO:26, 27, 28, 29, 30, 31, 32, 33, 34, 35, 36, 37, 38, 39, or 40; or an amino acid sequence at least 90% identical thereto.

In another aspect, the present disclosure provides a nucleic acid molecule, such as an expression construct, comprising a coding sequence for a recombinant polypeptide of the present disclosure. In some embodiments, the nucleic acid molecule further comprises a coding sequence for a chimeric antigen receptor (CAR). The CAR may target, for example, a tumor antigen such as AFP, BCMA, CD19, CD20, CD22, CD123, EpCAM, GPC2, GPC3, HER2, MUC16, ROR1, or ROR2. In further embodiments, the CAR may be bispecific, targeting, e.g., CD19 and CD20 or CD19 and CD22. In certain embodiments, the coding sequences for the recombinant polypeptide and the CAR are operably linked to the same promoter (e.g., a constitutive or inducible promoter; for example, an MND promoter) such that the two coding sequences are co-transcribed, and optionally the two coding sequences are separated by (i) an internal ribosome entry site (IRES) or (ii) a coding sequence for a self-cleaving peptide (e.g., a 2A peptide) wherein the coding sequences for the recombinant polypeptide, the CAR, and the self-cleaving peptide are in frame with each other.

In some embodiments, the nucleic acid molecule further comprises a coding sequence for a third polypeptide, optionally wherein the third polypeptide is human c-Jun or a functional analog thereof. In further embodiments, the coding sequences for the recombinant polypeptide, the CAR, and the human c-Jun are operably linked to the same promoter (e.g., a constitutive or inducible promoter; for example, an MND promoter) such that the three coding sequences are co-transcribed, and optionally the three coding sequences are separated from each other by (i) an IRES or (ii) a coding sequence for a self-cleaving peptide (e.g., a 2A peptide) wherein the coding sequences for the recombinant polypeptide, the CAR, the human c-Jun, and the self-cleaving peptide(s) are in frame with each other.

In some embodiments, the nucleic acid molecule is a viral vector, optionally a lentiviral or retroviral vector.

In other aspects, the present disclosure provides a cell (e.g., autologous or allogeneic human T cells) comprising the nucleic acid molecule described herein; a recombinant virion comprising the nucleic acid molecule; and a pharmaceutical composition comprising the cell, the nucleic acid molecule, or the virion, and a pharmaceutically acceptable carrier.

In another aspect, the present disclosure provides a method of treating a patient in need thereof, comprising administering the cell to the patient, optionally wherein the cell is autologous or allogeneic. In some embodiments, the patient has cancer, and is given the T cell preparation described herein, where the T cells express a CAR, a T cell receptor (TCR), an engineered TCR, or a TCR mimic that is specific for a tumor antigen present in the cancer. In further embodiments, the method comprises administering to the patient an effective amount of an antibody specific for human EGFR once the patient has been treated (e.g., the cancer has regressed), wherein the antibody elicits cytotoxicity against T cells expressing the recombinant polypeptide, and optionally the antibody is IgG₁ or IgG₂ (e.g., cetuximab).

The present disclosure also provides the cell, the nucleic acid molecule, the recombinant virus, the pharmaceutical composition for use in the treatment methods, as well as the cell, the nucleic acid molecule, or the virus for the manufacture of a medicament for treating a patient as described herein.

In yet another aspect, the present disclosure provides a method of making a genetically engineered human cell (e.g., engineered T cells), comprising providing an isolated human cell, and introducing the nucleic acid molecule or recombinant virus described herein into the human cell.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIGS. 1A and 1B show the binding of anti-EGFR antibody AY13 to live CAR+ T cells transduced with R12CAR-P2A-EGFRt or R12CAR-P2A-EGFRt-DEARKAIAR, or to total live untransduced cells. FIG. 1A: flow plot. FIG. 1B: a bar graph quantitating the geometric mean fluorescence intensity (gMFI) data from FIG. 1A. “DEARKAIAR”: a juxtamembrane sequence DEARKAIARVKRESKRIVEDAERLIREAAAASEKISREAERLI (SEQ ID NO:41). R12CAR: a CAR directed against ROR1. P2A: a self-cleaving peptide. EGFRt: a truncated human EGFR containing EGFR extracellular Domains III and IV and an EGFR transmembrane domain while lacking EGFR Domains I and II and EGFR intracellular sequence.

FIG. 2A depicts the domain structure of human EGFR from NCBI Reference Sequence NP_005219.2. The transmembrane domain is SEQ ID NO:5, and the juxtamembrane domain is SEQ ID NO:15 (full length sequence disclosed as SEQ ID NO:43). In the juxtamembrane domain, basic residues are indicated by red and “+,” and acidic residues by blue and “−.” Phosphorylated residues (green) are further described below.

FIG. 2B depicts the design of certain embodiments of the present EGFR-derived polypeptides (with transmembrane domain SEQ ID NO:5 without or with a juxtamembrane domain of RRR, SEQ ID NO:16, SEQ ID NO:12, or SEQ ID NO:13 (full length sequences disclosed as SEQ ID NOs:44-47, respectively, in order of appearance)). S. peptide: signal peptide. GMCSF: signal peptide derived from GM-CSF. Asterisks indicate the C-terminal end of the polypeptide.

FIGS. 3A-C show the expression of a series of bi-cistronic expression constructs for EGFR-derived polypeptides in primary T cells obtained from two donors and transduced with the expression constructs. FIG. 3A depicts the basic structure of the constructs, which include coding sequences for R12 CAR. FIGS. 3B and 3C show the gMFIs for bound ROR1-Fc fusion protein and AY13, respectively, in live R12 CAR+ transduced cells or live cells in the untransduced condition. Flow cytometry was performed eight days post transduction. FIGS. 3B and 3C disclose SEQ ID NOs:16, 12, 13, 16, 12, and 13, respectively, in order of appearance.

FIG. 4A is a bar graph showing the percentages of transduced T cells as indicated by R12 CAR expression. The T cells were those shown in FIGS. 3A-C. Flow cytometry was performed five days post transduction. FIG. 4A discloses SEQ ID NOs:16, 12, 13, 16, 12, and 13, respectively, in order of appearance.

FIG. 4B is a graph showing a comparison of EGFRt detection by Domain III-specific cetuximab and Domain III-specific AY13. Flow cytometry was performed five days post transduction. FIG. 4B discloses SEQ ID NOs:16, 12, and 13, respectively, in order of appearance.

FIGS. 5A and 5B are graphs showing the effects of transduction efficiency on R12 CAR and EGFRt surface expression, respectively, in primary T cells transduced with the bi-cistronic expression constructs of FIGS. 3A-C. Flow cytometry was performed four days post transduction. FIGS. 5A and 5B disclose SEQ ID NOs:16, 12, and 13, respectively, in order of appearance.

FIGS. 6A-C show the expression of a series of tri-cistronic expression constructs for EGFR-derived polypeptides in primary T cells obtained from two donors and transduced with the expression constructs. FIG. 6A depicts the basic structure of the constructs. FIGS. 6B and 6C show the gMFIs for bound ROR1-Fc fusion protein and AY13, respectively, in live R12 CAR+ transduced cells or total live untransduced cells. Flow cytometry was performed eight days post transduction. FIGS. 6B and 6C each disclose SEQ ID NO:16.

FIG. 7 shows the antibody-dependent cellular cytotoxicity (ADCC) induced by cetuximab in CAR-T cells expressing EGFR-derived polypeptides from the bi-cistronic constructs of FIG. 3A. The figure shows the fraction of CAR-T cells remaining after four hours of cetuximab treatment, relative to CAR-T cells not treated with the antibody. Ritux: rituximab.

FIG. 8 is a graph showing cetuximab-induced cytotoxicity in CAR-T cells expressing EGFR-derived polypeptides from the tri-cistronic constructs of FIG. 6A. The figure shows the fraction of CAR-T cells remaining after four hours of cetuximab treatment, relative to CAR-T cells not treated with the antibody. FIG. 8 discloses SEQ ID NO:16.

FIG. 9 shows cetuximab-induced cytotoxicity in CAR-T cells expressing EGFR-derived polypeptides from the tri-cistronic constructs of FIG. 6A. The figure shows the fraction of CAR-T cells remaining after 24 hours of cetuximab treatment, relative to CAR-T cells not treated with the antibody. FIG. 9 discloses SEQ ID NO:16.

FIG. 10 is a bar graph quantitating the gMFI for anti-EGFR antibody binding in live ROR1 CAR+ cells transduced with EGFRt or variants thereof having additional intracellular juxtamembrane sequences (R, RR, RRR, or RKR) or in total live cells in the untransduced condition.

FIGS. 11A and 11B show surface expression levels of EGFRt (white) or EGFR-RRR (EGFRt with an RRR juxtamembrane domain; grey) in mouse T cells transduced with MP71 retroviral constructs that were mono-cistronic (FIG. 11A) or bi-cistronic (FIG. 11B). MFI: mean fluorescence intensity.

FIG. 12A shows a schematic (top panel) of an in vivo study on the indicated CAR-T infusion products (middle panel) and expression levels of the EGFR polypeptides EGFRt or EGFR-RRR (bottom panel).

FIG. 12B is a panel of graphs showing the kinetics of circulating EGFRt and EGFR-RRR CAR-T cells following cetuximab (Cetx) treatment (white circle) compared to rituximab (control; Ritx) (black circle). Grey line represents depletion time point.

FIG. 12C shows the results of an in vivo study on the depletion kinetics of circulating EGFRt and EGFR-RRR transduced T cells. Top panel: a schematic showing the adoptive transfer of EGFR⁺ T cells. Bottom panel: graphs showing kinetics of circulating EGFR⁺ T cells following treatment with cetuximab (white circle). Grey line represents depletion time point.

FIG. 13A shows the results of an in vivo study on the depletion kinetics of circulating EGFRt (left) and EGFR-RRR (right) CAR-T cells following treatment with cetuximab (white circle) or vehicle (black circle). Shaded area represents post-depletion window.

FIG. 13B shows rebound kinetics of circulating B cells in the study shown in FIG. 13A. Two different doses of cetuximab were used as indicated.

FIG. 13C shows the frequency of B-cell-aplastic animals (below 3% CD19⁺ out of total CD45) following high (1 mg) or low (0.1 mg) dose cetuximab administration.

### DETAILED DESCRIPTION OF THE INVENTION

An important component of cell therapy is a compact, functionally inert cell surface marker that can be used for detecting, selecting, and enriching engineered cells, and for in vivo cell ablation. The present disclosure provides novel EGFR-derived proteins that can be used for these purposes. These proteins lack the ligand-binding and/or signal transduction functions of wildtype EGFR, but can still be recognized by common anti-EGFR antibodies.

Due to the design of their sequences, the present EGFR-derived proteins can be expressed at high levels on cell surface and therefore are particularly useful as a safety switch (suicide gene) in cell therapy. When the engineered cells in the therapy are no longer needed in the body, a pharmaceutical grade anti-EGFR antibody such as cetuximab, panitumumab, nimotuzumab, or necitumumab can be administered to the patient, thereby removing the engineered cells through antibody-dependent cellular cytotoxicity (ADCC), complement-dependent cytotoxicity (CDC), and/or antibody-dependent cellular phagocytosis (ADCP).

Unless otherwise indicated, EGFR as used herein refers to human EGFR. A human EGFR polypeptide sequence may be found at the UniProt database (Identifier No. P00533-1) and may have the following sequence:

| (SEQ ID NO: 1) | |
|---|---|
| 1 | MRPSGTAGAA LLALLAALCP ASRALEEKKV CQGTSNKLTQ LGTFEDHELS LQRMENNCEV |
| 61 | VLGNLEITYV QRNYDLSFLK TIQEVAGYVL IALNTVERIP LENLQIIRGN MYYENSYALA |
| 121 | VLSNYDANKT GLKELPMRNL QEILHGAVRF SNNPALCNVE SIQWRDIVSS DELSNMSMDF |
| 181 | QNHLGSCQKC DPSCPNGSCW GAGEENCQKL TKIICAQQCS GRCRGKSPSD CCHNQCAAGC |
| 241 | TGPRESDCLV CRKFRDEATC KDTCPPLMLY NPTTYQMDVN PEGKYSFGAT CVKKCPRNYV |
| 301 | VTDHGSCVRA CGADSYEMEE DGVRKCKKCE GPCRKVCNGI GIGEFKDSLS INATNIKHFK |
| 361 | NCTSISGDLH ILPVAFRGDS FTHTPPLDPQ ELDILKTVKE ITGFLLIQAW PENRTDLHAF |
| 421 | ENLEIIRGRT KQHGQFSLAV VSLNITSLGL RSLKEISDGD VIISGNKNLC YANTINWKKL |
| 481 | FGTSGQKTKI ISNRGENSCK ATGQVCHALC SPEGC [symbol] GPEP RDCVSCRNVS RGRECVDKCN |
| 541 | LLEGEPREFV ENSECIQCHP ECLPQAMNIT CTGRGPDNCI QCAHYIDGPH CVKTCPAGVM |
| 601 | GENNTLVWKY ADAGHVCHLC HPNCTYGCTG PGLEGCPTNG PKIPSIATGM VGALLLLLVV |
| 661 | ALGIGLEMRR RHIVRKRTLR RLLQERELVE PLTPSGEAPN QALLRILKET EFKKIKVLGS |
| 721 | GAFGTVYKGL WIPEGEKVKI PVAIKELREA TSPKANKEIL DEAYVMASVD NPHVCRLLGI |
| 781 | CLTSTVQLIT QLMPFGCLLD YVREHKDNIG SQYLLNWCVQ IAKGMNYLED RRLVHRDLAA |
| 841 | RNVLVKTPQH VKITDFGLAK LLGAEEKEYH AEGGKVPIKW MALESILHRI YTHQSDVWSY |
| 901 | GVTVWELMTF GSKPYDGIPA SEISSILEKG ERLPQPPICT IDVYMIMVKC WMIDADSRPK |
| 961 | FRELIIEFSK MARDPQRYLV IQGDERMHLP SPTDSNFYRA LMDEEDMDDV VDADEYLIPQ |
| 1021 | QGFFSSPSTS RTPLLSSLSA TSNNSTVACI DRNGLQSCPI KEDSFLQRYS SDPTGALTED |
| 1081 | SIDDTFLPVP EYINQSVPKR PAGSVQNPVY HNQPLNPAPS RDPHYQDPHS TAVGNPEYLN |
| 1141 | TVQPTCVNST FDSPAHWAQK GSHQISLDNP DYQQDFFPKE AKPNGIFKGS TAENAEYLRV |
| 1201 | APQSSEFIGA |

In the above sequence, the various EGFR domains are delineated as follows. The signal peptide spans amino acids 1-24. The extracellular sequence spans amino acids 25-645, wherein Domain I, Domain II, Domain III, and Domain IV span amino acids 25-188, 189-333, 334-504, and 505-645, respectively. The transmembrane domain spans amino acids 646-668. The intracellular domain spans amino acids 669-1,210, where the juxtamembrane domain spans amino acids 669-703 and the tyrosine kinase domain spans amino acids 704-1,210. Unless otherwise indicated, an EGFR amino acid position recited herein refers to the position in SEQ ID NO:1 or a corresponding position in a variant of SEQ ID NO:1 (e.g., a naturally occurring polymorphic variant or a genetically engineered variant).

### I. EGFR-Derived Polypeptides

The recombinant polypeptides of the present disclosure are derived from EGFR but contain only a partial, rather than entire, sequence of EGFR. These polypeptides are cell surface proteins when expressed in mammalian cells. The polypeptides' extracellular, transmembrane, and intracellular regions are described below.

### A. Extracellular Region

The extracellular region of the present EGFR-derived polypeptides comprises the epitope bound by an anti-EGFR antibody such as cetuximab. By way of example, the region may comprise Domain III of EGFR, such as the following Domain III sequence, or a functional variant thereof:

(SEQ ID NO: 2)
RKVCNGIGIG EFKDSLSINA TNIKHEKNCT SISGDLHILP
VAFRGDSFTH TPPLDPQELD ILKTVKEITG FLLIQAWPEN
RTDLHAFENL EIIRGRTKQH GQFSLAVVSL NITSLGLRSL
KEISDGDVII SGNKNLCYAN TINWKKLEGT SGQKTKIISN
RGENSCKATG Q

By “functional variant” is meant a sequence having sequence variations, such as deletions, insertions, and/or substitutions (e.g., conservative substitutions), that do not affect the sequence's desired biological function. A functional variant of SEQ ID NO:2 can be still bound by cetuximab.

To maintain the tertiary structure of the Domain III sequence, the extracellular region may further comprise additional EGFR sequences such as those that help stabilize disulfide bonds in the Domain III structure. For example, the extracellular region may comprise a Domain III sequence followed by a sequence derived from Domain IV of EGFR. A Domain IV-derived sequence may comprise the following Domain IV sequence:

(SEQ ID NO: 3)
VCHALCSPEG C [symbol] GPEPRDCV SCRNVSRGRE CVDKCNLLEG
EPREFVENSE CIQCHPECLP QAMNITCTGR GPDNCIQCAH
YIDGPHCVKT CPAGVMGENN TLVWKYADAG HVCHLCHPNC
TYGCTGPGLE GCPTNGPKIP S

A Domain IV-derived sequence may alternatively comprise a functional variant of SEQ ID NO:3. Such a functional variant can help maintain Domain III's tertiary structure to allow the binding of the polypeptide by an anti-EGFR antibody such as cetuximab. The functional variant may contain just a portion of a natural EGFR Domain IV, with or without additional sequences heterologous to EGFR (i.e., sequences that are not part of a natural EGFR sequence).

In some embodiments, a Domain IV-derived sequence includes a portion of a natural EGFR domain IV sequence, which portion includes amino acid residues involved in maintaining the structural fold of Domain III. Such amino acid residues include the W492 residue of mature EGFR (corresponding to W516 of SEQ ID NO:1 and W12 of SEQ ID NO:3; boxed in the sequences above) and optionally one or more residues adjacent to it. Structural analysis shows that W492 is important to the folding of EGFR Domain III, as this residue points into the core of Domain III and makes important side chain packing interactions. Examples of Domain IV-derived sequences are those including residues 492-496 of mature EGFR (corresponding to residues 516-520 of SEQ ID NO:1 and residues 12-16 of SEQ ID NO:3).

One particular example of a Domain IV-derived sequence is V [symbol] WGPEPGGGS (SEQ ID NO:4), in which natural Domain IV's residues 482-491 and 497-621 are removed, V481 is connected to W492 through a synthetic four-residue linker (boxed above), and P496 is followed by a G/S linker (underlined above) linking it to the transmembrane region of the present polypeptide.

In some embodiments, the extracellular region of the present EGFR-derived polypeptides lacks the EGFR extracellular portion that binds ligands such as EGF and TGF-alpha. For example, the extracellular region does not include any sequence of Domain I and/or Domain II of EGFR or includes only partial sequences from either or both Domains.

In some embodiments, the extracellular region of the present polypeptides includes additional sequences. For example, the extracellular region may comprise a stalk region immediately N-terminal to the transmembrane domain. The stalk region may be, for example, a flexible stalk such as a G/S rich peptide linker or a structured stalk such as the CH2-CH3 domains from an antibody constant region or an extracellular domain from another protein. The extracellular region also may comprise an additional functional domain, such as antigen-binding domains (e.g., an scFv or a designed ankyrin repeat protein (DARPin)).

### B. Transmembrane Region

The transmembrane region of the present polypeptides contains a hydrophobic sequence. This region may comprise an artificial sequence or may be derived from any transmembrane protein, which may be, for example, ERBB1 (EGFR), ERBB2 (HER2), ERBB3 (HER3), ERBB4 (HER4), INSR, IGF1R, INSRR, PGFRA, PGFRB, KIT, CSF1R, FLT3, VGFR1, VGFR2, VGFR3, FGFR1, FGFR2, FGFR3, FGFR4, PTK7, NTRK1, NTRK2, NTRK3, ROR1, ROR2, MUSK, MET, RON, UFO, TYRO3, MERTK, TIE1, TIE2, EPHA1, EPHA2, EPHA3, EPHA4, EPHA5, EPHA6, EPHA7, EPHA8, EPHAA, EPHB1, EPHB2, EPHB3, EPHB4, EPHB6, RET, RYK, DDR1, DDR2, ROS1, LMTK1, LMTK2, LMTK3, LTK, ALK, or STYK1.

One particular example of the transmembrane region is derived from EGFR, with the sequence of IATGMVGALLLLLVVALGIGLFM (SEQ ID NO:5), or a functional variant thereof.

### C. Intracellular Region

The inventors have unexpectedly discovered that inclusion of an appropriate juxtamembrane domain in the intracellular region of the EGFR-derived protein markedly increases the protein's cell surface expression level. A juxtamembrane domain refers to an intracellular portion of a cell surface protein immediately C-terminal to the transmembrane domain. A high cell surface expression level ensures that the cell expressing the protein is recognized by an anti-EGFR antibody and thus ensures the eradication of the cell through, e.g., ADCC, CDC, and/or ADCP.

The juxtamembrane domain in the present polypeptide may be from 1 to 20 (e.g., 2-20, 3-20, 4-20, 5-20, 2-18, 3-18, 4-18, or 5-18) amino acids long. They also can be longer than 20 amino acids. In some embodiments, the first 1 or more (e.g., first 2, 3, 4, 5, 6, 7, 8, 9, 10, 11, 12, 13, 14, 15, 16, 17, 18, 19, or 20) amino acids of the intracellular region of the present polypeptide is a net-neutral or net-positively charged sequence (e.g., the number of arginine and lysine residues is greater than or equal to the number of aspartic acid and glutamic acid residues). In further embodiments, those first amino acids contain more than 30% (e.g., more than 40, 50, 60, 70, 80, or 90%) hydrophilic amino acids. Non-limiting examples of amino acid choices at each position of the sequence appended to the C-terminus of the transmembrane domain are shown in Table 1 below.

TABLE 1

| Position | Possible amino acids |
|---|---|
| 1 | R, K, C, L, H, S, N, A, Y, F, M, W, G, T, Q |
| 2 | R, K, C, G, L, Q, I, Y, F, M, N, S, T, W, H |
| 3 | R, K, L, C, M, W, Y, I, N, V, T, Q, A, F, G, S, D, E, H |
| 4 | R, K, H, Q, G, S, C, N, V, W, P, F, T, D, E, Y |
| 5 | R, K, Q, C, G, A, I, L, N, P, T, W, S, D, E, Y, H |
| 6 | K, R, Q, P, V, D, N, Y, I, E, C, A, H, W, G, F, S, T |
| 7 | K, R, or another amino acid |
| 8 | K, R, S, Q, G, L, I, T, P, Y, N, A, F, W, D, H, E |
| 9 | K, R, G, L, Y, E, F, Q, S, A, H, P, T, N, D, W |
| 10 | G, A, E, R, D, K, T, Y, V, F, S, M, Q, L, N, P, W, H |
| 11 | K, R, Q, S, A, E, L, T, P, N, I, D, F, G, V, Y, W, H |
| 12 | Any amino acid |
| 13 | S, E, R, F, K, P, L, Y, D, or another amino acid |
| 14 | T, R, S, E, A, P, Q, K, N, V, or another amino acid |
| 15 | D, E, S, L, P, A, R, V, M, or another amino acid |
| 16 | E, V, Q, A, or another amino acid |
| 17 | E, L, D, Q, V, A, K, or another amino acid |
| 18 | Any amino acid |
| 19 | Any amino acid |
| 20 | E, G, L, R, S, V, Y, K, D, or another amino acid |

Some non-limiting examples of such juxtamembrane domains are shown below:

TABLE 2

| SEQ ID NO | Sequence | Net charge |
|---|---|---|
| n/a | K | +1 |
| n/a | KR | +2 |
| n/a | KRK | +3 |
| n/a | KSR | +2 |
| 6 | KSGSGS | +1 |
| n/a | SKR | +2 |
| 7 | KRSD | +1 |
| 8 | KRSDK | +2 |
| 9 | SGGGG | 0 |
| 10 | SGAGG | 0 |
| 11 | KRADK | +2 |
| 12 | RRRSGGGGSGGGGS | +3 |
| 13 | SGGGGSGGGGS | 0 |
| 14 | (GGGGS)n, n > 1 | 0 |

The present juxtamembrane domain may be derived from the juxtamembrane region of a natural cell surface protein, such as a juxtamembrane region (e.g., the entire or partial sequence of the first 20 juxtamembrane amino acids) of a human receptor tyrosine kinase that interacts with phosphatidylcholine (PC), phosphatidylserine (PS), or phosphatidylinositol-4,5-bisphosphate (PIP2) (see, e.g., Hedger et al., Sci Rep. (2015) 5:9198). Examples of receptor tyrosine kinases are ERBB1 (EGFR), ERBB2 (HER2), ERBB3 (HER3), ERBB4 (HER4), INSR, IGF1R, INSRR, PGFRA, PGFRB, KIT, CSF1R, FLT3, VGFR1, VGFR2, VGFR3, FGFR1, FGFR2, FGFR3, FGFR4, PTK7, NTRK1, NTRK2, NTRK3, ROR1, ROR2, MUSK, MET, RON, UFO, TYRO3, MERTK, TIE1, TIE2, EPHA1, EPHA2, EPHA3, EPHA4, EPHA5, EPHA6, EPHA7, EPHA8, EPHAA, EPHB1, EPHB2, EPHB3, EPHB4, EPHB6, RET, RYK, DDR1, DDR2, ROS1, LMTK1, LMTK2, LMTK3, LTK, ALK, and STYK1. If desired, the derived sequence may contain mutations (e.g., substitutions or deletions) that remove residues known to be phosphorylated so as to circumvent any unintended signal transducing ability of the present protein.

In some embodiments, the juxtamembrane domain of the present polypeptide comprises a juxtamembrane region of EGFR, such as:

(SEQ ID NO: 15)
RRRHIVRKR [symbol] LRRLLQERELVEPL [symbol] P [symbol] GEAPNQAL

In some embodiments, an EGFR-derived juxtamembrane domain is derived from the first 19 amino acids of a natural EGFR juxtamembrane region (e.g., SEQ NO ID:15) and does not include the entirety of the remaining portion of the natural juxtamembrane region, so as to avoid dimerization of the present polypeptide. In some embodiments, the residues known to be phosphorylated (boxed above, corresponding to T678, T693, and S695 of SEQ ID NO:1) are deleted or substituted. Nonlimiting examples of EGFR-derived juxtamembrane domains comprise one of the following sequences:

TABLE 3

| SEQ ID NO | Sequence | Net Charge |
|---|---|---|
| 16 | RRRHIVRKR | +6 |
| 17 | RRRHIVRK | +5 |
| 18 | RRRHIVR | +4 |
| 19 | RRRHIV | +3 |
| 20 | RRRHI | +3 |
| 21 | RRRH | +3 |
| n/a | RRR | +3 |
| n/a | RKR | +3 |
| n/a | RR | +2 |
| n/a | R | +1 |

In some embodiments, the intracellular region also includes an additional sequence C-terminal to the juxtamembrane domain, e.g., a functional domain (e.g., a switch receptor).

The present EGFR-derived protein lacks a functional tyrosine kinase domain of EGFR such that the protein lacks signal transducing ability. For example, the protein lacks the entirety of a region that corresponds to amino acids 704-1,210 of SEQ ID NO:1. In some embodiments, the intracellular region does not contain any potential phosphorylation motif.

### D. Signal Peptide

In some embodiments, the coding sequence for the present polypeptide includes a coding sequence for a signal peptide. The signal peptide may facilitate the cell surface expression of the polypeptide and is cleaved from the mature polypeptide. The signal peptide may be derived from that of any cell surface protein or secreted protein. For example, the signal peptide may be a signal peptide shown below:

TABLE 4

| SEQ ID NO | Source | Sequence |
|---|---|---|
| 22 | EGFR | MRPSGTAGAALLALLAALCPASRA |
| 23 | GM-CSF | MLLLVTSLLLCELPHPAFLLIP |
| 24 | human Ig kappa | MVLQTQVFISLLLWISGAYG |
| 25 | human CD33 | MPLLLLLPLLWAGALA |

The various domains described above for the extracellular, transmembrane, and intracellular regions of the present polypeptides may be linked directly or through a peptide linker.

### E. Examples of EGFR-Derived Polypeptides

In some embodiments, the present polypeptide comprises, consists of, or consists essentially of EGFR Domain III (italicized), Domain IV (underlined), and transmembrane domain, with a juxtamembrane domain (not shown) appended to the C-terminus of the transmembrane domain, with or without a signal peptide (not shown):

(SEQ ID NO: 26)
RKVCNGIGIGEFKDSLSINATNIKHFKNCTSISGDLHILPVAFRGDSFTHTPPLDPQELDILKTV
KEITGELLIQAWPENRTDLHAFENLEIIRGRTKQHGQFSLAVVSLNITSLGLRSLKEISDGDVII
SGNKNLCYANTINWKKLFGTSGQKTKIISNRGENSCKATGQVCHALCSPEGCWGPEPRDCVSCRN
VSRGRECVDKCNLLEGEPREFVENSECIQCHPECLPQAMNITCTGRGPDNCIQCAHYIDGPHCVK
TCPAGVMGENNTLVWKYADAGHVCHLCHPNCTYGCTGPGLEGCPTNGPKIPSIATGMVGALLLLL
VVALGIGLFM

In some embodiments, the present polypeptide comprises, consists of, or consists essentially of EGFR Domain III (italicized), [symbol] (boldfaced and underlined), EGFR transmembrane domain, and a juxtamembrane domain (not shown) appended to the C-terminus of the transmembrane domain, with or without a signal peptide (not shown):

(SEQ ID NO: 27)
RKVCNGIGIGEFKDSLSINATNIKHEKNCTSISGDLHILPVAFRGDSFTHTPPLDPQELDILKTV
KEITGELLIQAWPENRTDLHAFENLEIIRGRTKQHGQFSLAVVSLNITSLGLRSLKEISDGDVII
SGNKNLCYANTINWKKLFGTSGQKTKIISNRGENSCKATGQVCHALCSPEGCWGPEPRDCVSGGP
SIATGMVGALLLLLVVALGIGLFM

In some embodiments, the present polypeptide comprises, consists of, or consists essentially of EGFR Domain III (italicized), [symbol] (boldfaced and underlined), EGFR transmembrane domain, and a juxtamembrane domain (not shown) appended to the C-terminus of the transmembrane domain, with or without a signal peptide (not shown):

(SEQ ID NO: 28)
RKVCNGIGIGEFKDSLSINATNIKHFKNCTSISGDLHILPVAFRGDSFTHTPPLDPQELDILKTV
KEITGFLLIQAWPENRTDLHAFENLEIIRGRTKQHGQFSLAVVSLNITSLGLRSLKEISDGDVII
SGNKNLCYANTINWKKLFGTSGQKTKIISNRGENSCKATGQTGSGWGPEPGGGSPSIATGMVGAL
LLLLVVALGIGLFM

In some embodiments, the present polypeptide comprises, consists of, or consists essentially of a [symbol] (boldfaced), EGFR Domain III (italicized), EGFR domain IV (underlined), EGFR transmembrane domain (boldfaced and italicized) and a juxtamembrane domain having the sequence of RRR:

(SEQ ID NO: 29)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTYGCTGPGLEGC
PTNGPKIPSIATGMVGALLLLLVVALGIGLFMRRR

In some embodiments, the present polypeptide comprises, consists of, or consists essentially of a [symbol] (boldfaced), EGFR Domain III (italicized), EGFR Domain IV (underlined), EGFR transmembrane domain (boldfaced and italicized), and a juxtamembrane domain having the sequence of RRRHIVRKR (SEQ ID NO:16):

(SEQ ID NO: 30)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTYGCTGPGLEGC
PTNGPKIPSIATGMVGALLLLLVVALGIGLFMRRRHIVRKR

In some embodiments, the present polypeptide comprises, consists of, or consists essentially of a [symbol] (boldfaced), EGFR Domain III (italicized), EGFR Domain IV (underlined), EGFR transmembrane domain (boldfaced and italicized), and a juxtamembrane domain having the sequence of RRRSGGGGSGGGGS (SEQ ID NO:12):

(SEQ ID NO: 31)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTYGCTGPGLEGC
PTNGPKIPSIATGMVGALLLLLVVALGIGLFMRRRSGGGGSGGGGS

In some embodiments, the present polypeptide comprises, consists of, or consists essentially of a [symbol] (boldfaced), EGFR Domain III (italicized), EGFR Domain IV (underlined), EGFR transmembrane domain (boldfaced and italicized), and a juxtamembrane domain having the sequence of SGGGGSGGGGS (SEQ ID NO:13)

(SEQ ID NO: 32)
MLLLVTSLLLCELPHPAFLLIPRKVCNGIGIGEFKDSLSINATNIKHEKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECLPQAMNITC
TGRGPDNCIQCAHYIDGPHCVKTCPAGVMGENNTLVWKYADAGHVCHLCHPNCTYGCTGPGLEGC
PTNGPKIPSIATGMVGALLLLLVVALGIGLFMSGGGGSGGGGS

In some embodiments, the present polypeptide comprises, consists of, or consists essentially of a [symbol] (boldfaced), EGFR Domain III (italicized), modified portion of EGFR Domain IV (underlined), EGFR transmembrane domain (boldfaced and italicized), and a juxtamembrane domain having the sequence of RRR:

(SEQ ID NO: 33)
MLLLVTSLLLCELPHPAFLLIPRKVCNGIGIGEFKDSLSINATNIKHEFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSGGPSIATGMVGALLLLLVVALGIGLFMRRR

In some embodiments, the present polypeptide comprises, consists of, or consists essentially of a [symbol] (boldfaced), EGFR Domain III (italicized), modified portion of EGFR Domain IV (underlined), EGFR transmembrane domain (boldfaced and italicized), and a juxtamembrane domain having the sequence of RRRHIVRKR (SEQ ID NO:16)

(SEQ ID NO: 34)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSGGPSIATGMVGALLLLLVVALGIGLFMRRRHIVRKR

In some embodiments, the present polypeptide comprises, consists of, or consists essentially of a [symbol] (boldfaced), EGFR Domain III (italicized), modified portion of EGFR Domain IV (underlined), EGFR transmembrane domain (boldfaced and italicized), and a juxtamembrane domain having the sequence of RRRSGGGGSGGGGS (SEQ ID NO:12):

(SEQ ID NO: 35)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSGGPSIATGMVGALLLLLVVALGIGLFMRRRSGGGGSGGGGS

In some embodiments, the present polypeptide comprises, consists of, or consists essentially of a [symbol] (boldfaced), EGFR Domain III (italicized), modified portion of EGFR Domain IV (underlined), EGFR transmembrane domain (boldfaced and italicized), and a juxtamembrane domain having the sequence of SGGGGSGGGGS (SEQ ID NO:13):

(SEQ ID NO: 36)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQVC
HALCSPEGCWGPEPRDCVSGGPSIATGMVGALLLLLVVALGIGLFMSGGGGSGGGGS

In some embodiments, the present polypeptide comprises, consists of, or consists essentially of a [symbol] (boldfaced), EGFR Domain III (italicized), synthetic sequence (underlined), EGFR transmembrane domain (boldfaced and italicized), and a juxtamembrane domain having the sequence of RRR:

(SEQ ID NO: 37)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQTG
SGWGPEPGGGSPSIATGMVGALLLLLVVALGIGLFMRRR

In some embodiments, the present polypeptide comprises, consists of, or consists essentially of a [symbol] (boldfaced), EGFR Domain III (italicized), synthetic sequence (underlined), EGFR transmembrane domain (boldfaced and italicized), and a juxtamembrane domain having the sequence of RRRHIVRKR (SEQ ID NO:16):

(SEQ ID NO: 38)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQTG
SGWGPEPGGGSPSIATGMVGALLLLLVVALGIGLFMRRRHIVRKR

In some embodiments, the present polypeptide comprises, consists of, or consists essentially of a [symbol] (boldfaced), EGFR Domain III (italicized), synthetic sequence (underlined), EGFR transmembrane domain (boldfaced and italicized), and a juxtamembrane domain having the sequence of RRRSGGGGSGGGGS (SEQ ID NO:12):

(SEQ ID NO: 39)
MLLLVTSLLLCELPHPAFLLIPRKVCNGIGIGEFKDSLSINATNIKHEKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQTG
SGWGPEPGGGSPSIATGMVGALLLLLVVALGIGLFMRRRSGGGGSGGGGS

In some embodiments, the present polypeptide comprises, consists of, or consists essentially of a [symbol] (boldfaced), EGFR Domain III (italicized), synthetic sequence (underlined), EGFR transmembrane domain (boldfaced and italicized), and a juxtamembrane domain having the sequence of SGGGGSGGGGS (SEQ ID NO:13):

(SEQ ID NO: 40)
MLLLVTSLLLCELPHPAFLLIPRKVCNGIGIGEFKDSLSINATNIKHFKNCTSISGDLHILPVAF
RGDSFTHTPPLDPQELDILKTVKEITGELLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQTG
SGWGPEPGGGSPSIATGMVGALLLLLVVALGIGLFMSGGGGSGGGGS

Also provided in the present disclosure are EGFR-derived polypeptides that are at least 90% (e.g., at least 91, 92, 93, 94, 95, 96, 97, 98, or 99%) identical in sequence to the above exemplified sequences.

### II. Expression Constructs for the EGFR-Derived Proteins

The present disclosure provides expression constructs suitable for expressing the EGFR-derived proteins in cells that are used in cell therapy. An expression construct of the present disclosure includes an expression cassette comprising a coding sequence for the EGFR-derived polypeptide (preferably including a signal peptide) linked operably to one or more transcriptional regulatory elements. As used herein, “transcriptional regulatory elements” refer to nucleotide sequences in the expression construct that control expression of the coding sequence, for example, by regulating the tissue-specific expression patterns and transcription efficiency of the EGFR-derived polypeptide coding sequence, the stability of the RNA transcripts, and the translation efficiency of the RNA transcripts. These elements may be one or more of a promoter, a Kozak sequence, an enhancer, an RNA-stabilizing element (e.g., a WPRE sequence), a polyadenylation signal, and any combination thereof.

In some embodiments, the expression cassette contains a mammalian promoter that is constitutively active or inducible in the target cells. Examples of useful promoters are, without limitation, a Moloney murine leukemia virus (MoMuLV) LTR, an MND (a synthetic promoter containing the U3 region of a modified MoMuLV LTR with myeloproliferative sarcoma virus enhancer), a Rous sarcoma virus (RSV) LTR, a cytomegalovirus (CMV) promoter, a CMV immediate early promoter, a simian virus 40 (SV40) promoter, a dihydrofolate reductase (DHFR) promoter, a β-actin promoter, a phosphoglycerate kinase (PGK) promoter, an EFlα promoter, a thymidine kinase (TK) promoter, a tetracycline responsive promoter (TRE), an E2 factor (E2F) promoter, the human telomerase reverse transcriptase (hTERT) promoter, and an RU-486-responsive promoter.

In certain embodiments, the expression cassette also comprises additional regulatory sequences, for example, an internal ribosome entry site (IRES) or a sequence encoding a self-cleaving peptide to allow co-expression of another polypeptide in addition to the EGFR-derived polypeptide. Examples of self-cleaving peptides (also known as ribosomal skipping peptides) are 2A peptides, which are viral derived peptides with a typical length of 18-22 amino acids and include T2A, P2A, E2A, and F2A (Liu et al., Sci Rep. (2017) 7:2193).

In some embodiments, the present expression construct also expresses an antigen receptor and/or another additional polypeptide. The antigen receptor may be, for example, an antibody, an engineered antibody such as an scFv, a CAR, an engineered TCR, a TCR mimic (e.g., an antibody-T cell receptor (abTCR) or a chimeric antibody-T cell receptor (caTCR)), or a chimeric signaling receptor (CSR). By way of example, an abTCR may comprise an engineered TCR in which the antigen-binding domain of a TCR (e.g., an alpha/beta TCR or a gamma/delta TCR) has been replaced by that of an antibody (with or without the antibody's constant domains); the engineered TCR then becomes specific for the antibody's antigen while retaining the TCR's signaling functions. A CSR may comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ). See, e.g., EP340793B1, WO 2017/070608, WO 2018/200582, WO 2018/200583, WO 2018/200585, and Xu et al., Cell Discovery (2018) 4:62.

The antigen receptor may target an antigen of interest (e.g., a tumor antigen or an antigen of a pathogen). The antigens may include, without limitation, AFP (alpha-fetoprotein), αvβ6 or another integrin, BCMA, B7-H3, B7-H6, CA9 (carbonic anhydrase 9), CCL-1 (C-C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66c, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In some embodiments, the antigen receptor may be bispecific and target two different antigens, such as two of the antigens listed above. For example, the antigen receptor, such as a CAR, targets CD19 and CD20, or CD19 and CD22.

The additional polypeptide may be, for example, a cytokine (e.g., IL-2, IL-7, IL-12, IL-15, IL-23, and engineered variants thereof), a cytokine receptor (e.g., IL-12R, IL-7R, and engineered variants thereof), a chemokine, a transcription factor (e.g., c-Jun or c-fos; see, e.g., WO 2019/118902), functional analogs thereof, other engineered receptors (e.g. TGFBetaR), and other engineered effectors (e.g., secretory secondary effector; see, e.g., WO 2018/200585). By “functional analog” is meant a molecule that has the same or similar biological activity of interest as the cognate polypeptide or peptide even though there are sequence differences between it and the cognate molecule.

The coding sequences of these additional polypeptides may be under the control of different promoters from the EGFR-derived polypeptide coding sequence. Alternatively, they may be under the control of the same promoter as the EGFR-derived coding sequence but are separated from each other through an IRES or an in-frame coding sequence for a 2A peptide, such that the coding sequences can be co-expressed under the same promoter.

The expression constructs of the present disclosure may be delivered to target cells in vitro, ex vivo or in vivo by suitable means such as electroporation, sonoporation, viral transduction, lipofection, microinjection, biolistics, virosomes, liposomes, immunoliposomes, and nanoparticles (e.g., polymeric or lipid nanoparticles). In some embodiments, the expression constructs may be viral vectors and are delivered to the target cells through recombinant viruses containing the constructs. The viral vectors contain the EGFR-derived polypeptide expression cassette and minimal viral sequences required for packaging and subsequent integration into a host (if applicable). The missing viral functions are supplied in trans by the packaging cell line used to package the recombinant virus. The viral vector may be, for example, vaccinia vectors, adenoviral vectors, lentiviral vectors, poxyviral vectors, herpes simplex viral vectors, adeno-associated viral vectors, retroviral vectors, and hybrid viral vectors. In part depending on virus type, the EGFR-derived polypeptide expression cassette may be stably integrated into the genome of the target cells, or remain in the cells episomally. Integration into the host genome is possible with retrovirus and lentivirus.

### III. Pharmaceutical Use of Cells Expressing the EGFR-Derived Proteins

The present expression constructs may be introduced into cells used in cell therapy. These cells are, for example, multipotent cells such as hematopoietic stem cells, various progenitor or precursor cells of hematopoietic lineages, and various immune cells (e.g., human autologous or allogeneic T, natural killer (NK), dendritic, or B cells). These cells may also be pluripotent stem cells (PSCs) such as human embryonic stem cells and induced PSCs, which can be used to generate therapeutic cell populations. In some embodiments, pluripotent and multipotent cells are differentiated into a desired cell type in vitro before being implanted into the patient.

In some embodiments, the present disclosure provides engineered T lymphocytes that express the EGFR-derived protein and from the same construct or from a separate construct, one or more additional polypeptides. The one or more additional polypeptides may be an antigen receptor such as an antibody, an engineered antibody such as an scFv, a CAR, an engineered TCR, a TCR mimic (e.g., an abTCR or caTCR), or a CSR, as described above. The antigen receptor may target, for example, the antigens described above. The additional polypeptide also may be, for example, a cytokine, a cytokine receptor, a chemokine, a transcription factor, a functional analog of the foregoing, another engineered receptor, or an engineered effector as described above. The coding sequences of these additional polypeptides may be under the control of different promoters from the EGFR-derived polypeptide coding sequence. In some embodiments, the present disclosure provides engineered autologous or allogeneic NK cells expressing engineered receptors, and engineered B lymphocytes expressing an antibody, an engineered antibody, or an engineered tissue-specific cell expressing a therapeutic protein.

The genetically engineered cells described herein may be provided in a pharmaceutical composition containing the cells and a pharmaceutically acceptable carrier. The pharmaceutically acceptable carrier may be cell culture medium that optionally does not contain any animal-derived component. For storage and transportation, the cells may be cryopreserved. Prior to use, the cells may be thawed, and diluted in a sterile cell medium. The cells may be administered into the patient systemically (e.g., through intravenous injection or infusion), or locally (e.g., through direct injection to a local tissue, e.g., at the site of a solid tumor).

A therapeutically effective number of engineered cells are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. For example, a therapeutically effective amount of engineered CAR-T cells is an amount that is sufficient to cause tumor growth arrest, tumor regression, prevention of tumor metastasis, or prevention of tumor recurrence.

Once the engineered cells are no longer desired in a patient, e.g., when the cells do not function properly or when the therapeutic goal has been achieved, an anti-EGFR antibody may be administered to the patient at an amount that is sufficient to cause antibody-mediated killing of the cells. For example, cetuximab (e.g., Erbitux®) can be administered through infusion at one or more doses determined as appropriate for the number of engineered cells remaining in the patient.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to the manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

### EXAMPLES

In order for the present disclosure to be better understood, the following examples are set forth. These examples are for illustration only and are not to be construed as limiting the scope of the present disclosure in any manner.

### Example 1: Surface Expression of EGFR-Derived Polypeptides

This example describes studies analyzing the effects of various juxtamembrane domains on the cell surface expression levels of EGFR-derived polypeptides.

### Methods

### Expression Constructs

Lentiviral constructs were generated with bi-cistronic or tri-cistronic expression cassettes. In constructs with bi-cistronic expression cassettes, the coding sequences for (i) a ROR1-specific R12 CAR, (ii) a P2A self-cleaving peptide, and (iii) EGFRt (a truncated EGFR having only Domains III and IV and the transmembrane domain; SEQ ID NO:26) or a variant having additionally an intracellular juxtamembrane domain were linked in frame and placed under the control of an MND promoter. In constructs with tri-cistronic expression cassettes, the coding sequences for (i) c-Jun, (ii) a P2A peptide, (iii) a ROR1-specific R12 CAR, (iv) a P2A peptide; and (v) EGFRt or a variant having additionally an intracellular juxtamembrane domain were linked in frame and placed under the control of an MND promoter. The R12 CAR was derived from the R12 anti-ROR1 antibody (Yang et al., PLoS One. (2011) 6:e21018) and contains a CD28-derived transmembrane domain, a 4-1BB costimulatory domain, and a CD3 zeta signaling domain.

### Cell Culture and Lentiviral Transduction

Jurkat cells were obtained from American Type Culture Collection (ATCC; Manassas VA). For lentiviral transduction, the cells were fed with fresh media 4-16 hours before transduction, followed by incubation with lentivirus in complete media+LentiBOOST™ at the manufacturer's recommended concentration (Sirion Biotech). Eighteen hours after transduction, lentivirus and LentiBOOST™ were diluted by addition of 1 volume of fresh media.

Pre-selected, cryopreserved primary human CD4+ and CD8+ T cells from normal donors were obtained from Bloodworks (Seattle WA). Human T cells were cultured in OpTmizer medium (Thermo Fisher) supplemented with Immune Cell Serum Replacement (Thermo Fisher), 2 mM L-glutamine (Gibco), 2 mM Glutamax (Gibco), 200 IU/ml IL-2 (R&D systems), 120 IU/ml IL-7 (R&D systems), and 20 IU/ml IL-15 (R&D systems). For lentiviral transduction, the T cells were stimulated with a 1:100 dilution of T cell TransAct (Miltenyi) for 30 hours. Virus was then added to the T cells for 18-24 hours. Stimulation and viral infection were then terminated by addition of 7 volumes of fresh media without TransAct, and cells were cultured for 3-7 additional days before analysis.

### Flow Cytometry

Flow cytometry was performed on a Ze5 cytometer (Bio-Rad Laboratories). To determine expression of cell surface markers, about 1×10⁵ to 2×10⁵ total cells were transferred to a V bottom 96 well culture dish (Corning). Cells were washed twice with flow cytometry staining buffer (eBioscience), and then stained with the relevant reagents in a total volume of 50 μl flow cytometry staining buffer for 30 minutes on ice. After staining, the cells were washed twice with flow cytometry staining buffer, fixed in FluoroFix Buffer (BioLegend), and kept at 4° C. in the dark until analysis. Flow cytometry data was analyzed using FlowJo 10 (Tree Star).

For flow cytometry analysis, AY13 antibody labeled with fluorochrome BV421 (BioLegend) was used to detect EGFR variants. Purified recombinant ROR1 fused to human Ig Fc was produced in-house and conjugated to Alexa 647 dye for detecting R12 CAR. Fluor 780 Fixable Viability dye (eBioscience) was included during primary antibody stain at a 1:8000 dilution.

### Results

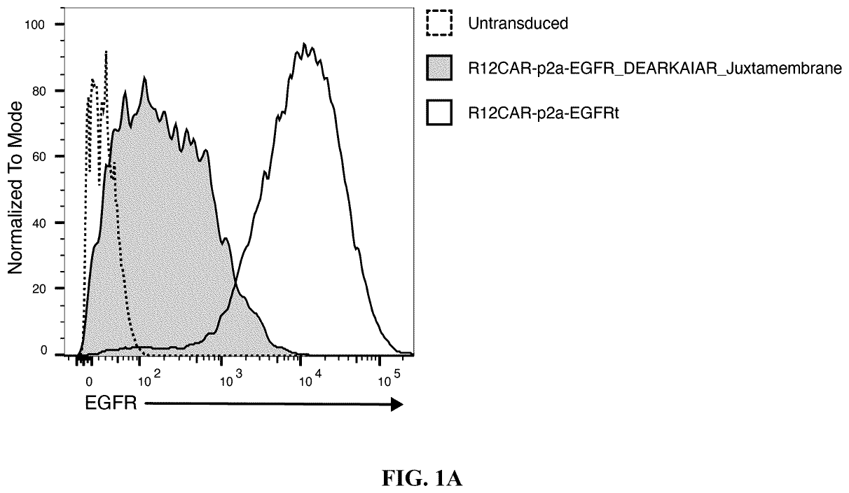

To modulate the cell surface expression of EGFRt, we fused it at the transmembrane C-terminus to a 43 amino acid synthetic sequence (DEARKAIARVKRESKRIVEDAERLIREAAAASEKISREAERLI; SEQ ID NO:41), which contains two acidic residues (aspartic acid and glutamic acid) proximal to the membrane. We found that the cell surface detection of EGFRt was dramatically reduced as compared to EGFRt without the C-terminal fusion in transduced primary T cells (FIGS. 1A and 1B). By contrast, when we fused EGFRt at the transmembrane C-terminus to (G₄S)₂ (SEQ ID NO:42, where n=2), the fusion protein was detected at markedly increased (five-fold) levels at the cell surface as compared to EGFRt without the fusion ((Jurkat cells; data not shown). These data suggest that inclusion of an appropriate juxtamembrane intracellular segment could modulate the surface expression of EGFRt variants.

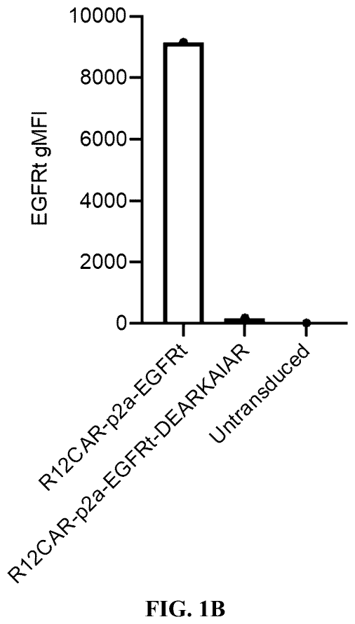

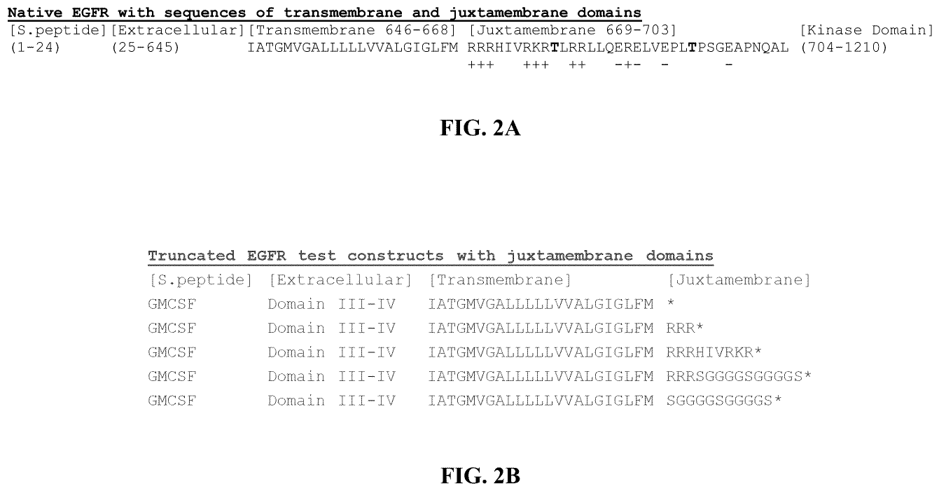

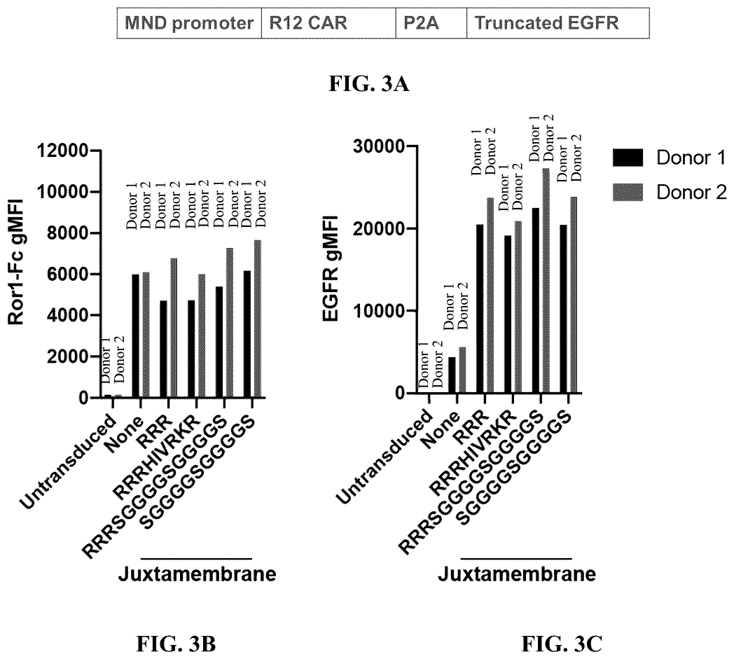

We hypothesized that certain amino acid compositions in the juxtamembrane intracellular region could increase EGFRt marker surface expression by enhancing membrane insertion during protein synthesis and/or improved stability of the transmembrane protein. To test this hypothesis, we generated EGFRt modules containing EGFR Domains III-IV, EGFR transmembrane domain, and short intracellular domains derived from the native EGFR sequence or synthetic sequences. Since T678 of human EGFR may be a site of regulatory phosphorylation, we selected for testing proteins containing amino acids 669-671 (RRR) or 669-677 (RRRHIVRKR; SEQ ID NO:16) (FIGS. 2A and 2B). Since glycine may disrupt α-helical structure and is enriched in the juxtamembrane region of other receptor tyrosine kinase proteins, we also tested intracellular domains containing unstructured glycine/serine-rich linkers (SGGGGSGGGGS; SEQ ID NO:13), or a short portion of the native juxtamembrane domain followed by a glycine/serine-rich linker (RRRSGGGGSGGGGS; SEQ ID NO:12). For this study, primary T cells were transduced with lentivirus expressing the ROR1-specific R12 CAR linked by a P2A skip sequence to the EGFRt variants (FIG. 3A). The expression of R12 CAR and the co-expressed EGFR-derived polypeptides was measured by flow cytometry.

The data show that, with the exception of a juxtamembrane sequence comprising membrane-proximal acidic residues (FIGS. 1A and 1B), EGFRt variants containing a juxtamembrane sequence consistently demonstrated higher mean fluorescence intensity, as compared to an EGFRt polypeptide without a juxtamembrane sequence (FIG. 3B). Notably, surface expression of the translationally linked R12 CAR was consistent for all constructs (FIG. 3B), indicating a direct impact of the juxtamembrane sequence on EGFRt stability rather than an effect on modulation of mRNA stability or translation efficiency, which would have impacted both CAR and EGFRt expression.

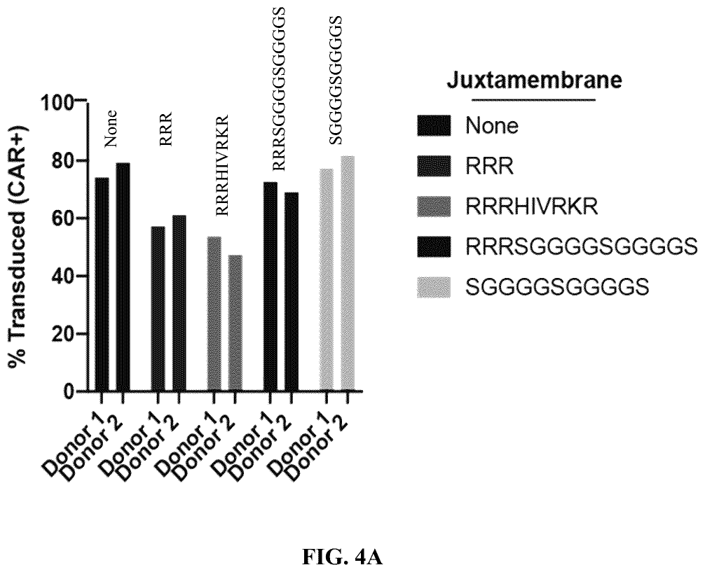

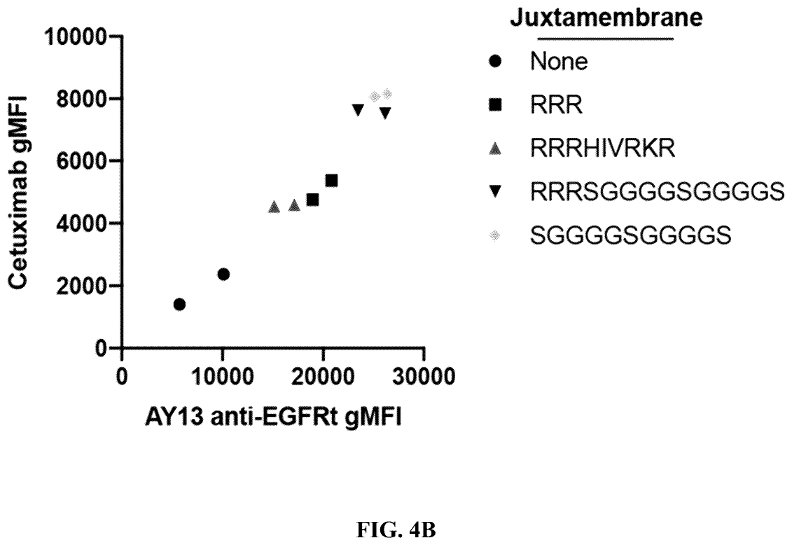

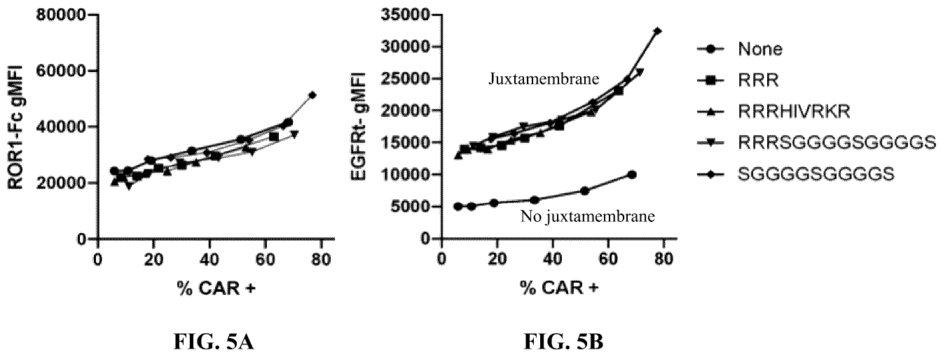

To confirm that binding of the EGFR Domain III specific AY13 monoclonal antibody accurately reflected cetuximab binding, we compared EGFRt expression levels (gMFI) determined by AY13 vs. a cetuximab biosimilar. The data demonstrated a linear relationship between gMFIs of the two antibodies and a clear increase in the cetuximab biosimilar's binding to EGFRt variants having a juxtamembrane sequence (FIGS. 4A and 4B). To investigate the role of viral copy number on EGFRt expression, we transduced primary T cells over a range of lentiviral titers. Similar to previous results, the surface gMFI of the R12 CAR was uniform across all constructs at a matching transduction frequency. In contrast, the surface gMFI of all EGFRt variants containing an intracellular sequence was about three to five-fold higher than that of the EGFRt marker lacking this sequence (FIGS. 5A and 5B). These data indicate that the presence of a suitable intracellular domain boosts the cell surface display of the EGFRt protein despite comparable translation efficiency, as measured by the expression level of the co-expressed R12 CAR. The results show that enhanced membrane insertion during protein synthesis or improved membrane protein stability plays a role in improving surface expression of the EGFR-derived proteins.

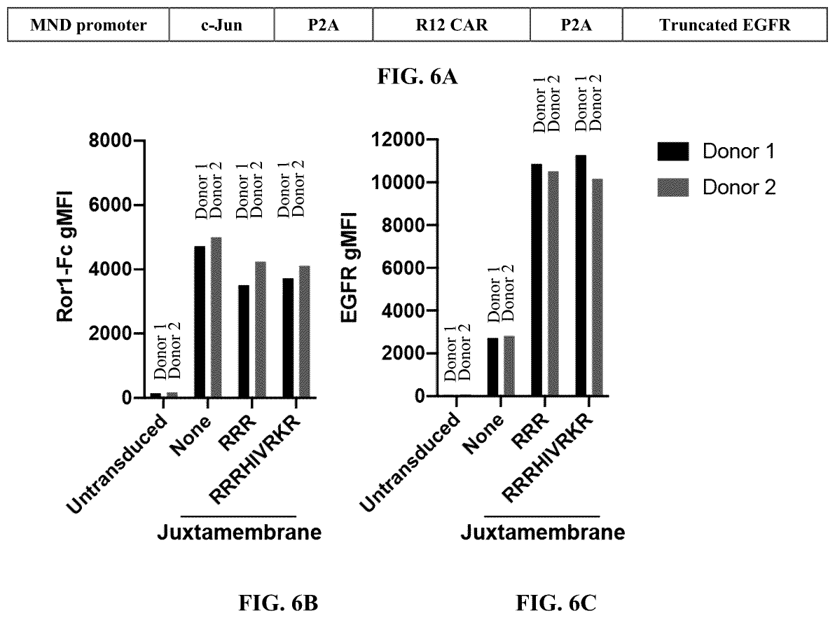

As the number of cistronic elements in a 2A-containing expression cassette increases, there is a general pattern of decreased expression of those elements from 5′ to 3′, driven in part by ribosome drop-off and inefficient cleavage of 2A elements. For this reason, maximizing effective surface expression of EGFRt variants is particularly important for tri-cistronic and higher order vectors. To test the impact of juxtamembrane sequences on EGFRt expression in a tri-cistronic expression cassette, we generated lentiviral vectors comprising the c-Jun transcription factor, R12 CAR, and an EGFRt module linked by P2A skip sequences (FIG. 6A). The EGFRt modules contained either no intracellular domain or an intracellular domain comprising amino acids 669-671 or 669-677 from the human EGFR sequence. As observed for the bi-cistronic vector, R12 CAR expression was similar across all constructs. In contrast, the surface expression of EGFRt increased by about four folds with the addition of a juxtamembrane sequence (FIGS. 6B and 6C).

### Example 2: Cetuximab-Mediated ADCC of CAR-T cells Expressing EGFRt Variants

This example describes studies analyzing the efficiency of the EGFR-derived proteins described herein as a safety switch in cell therapy. Altered surface expression of EGFRt could impact the utility of this marker as a selection marker and safety switch in vivo. Cetuximab induces ADCC of tumor cells in an EGFR-dependent manner (Kimura et al., Cancer Sci. (2007) 98(8):1275-80). As all four juxtamembrane sequences tested induced a similar increase in surface EGFRt expression, we selected EGFRt constructs containing no juxtamembrane sequence or containing the human EGFR derived RRR and RRRHIVRKR (SEQ ID NO:16) sequences for ADCC testing.

### Methods

Primary human natural killer (NK) cells were used as effector cells in ADCC assays. Natural killer cells were isolated from cryopreserved, T cell depleted (CD4−/CD8−) PBMC (AllCells) by negative selection using the EasySep Human NK Cell Kit (StemCell) according to manufacturer's protocol. To activate their cytolytic function, isolated NK cells were cultured in RPMI-10 supplemented with 10 ng/ml human IL-15 overnight before use (Wagner et al., J Clin Invest. (2017) 127(11):4042-58; Derer 2012, J Immunol. (2012) 189(11):5230-9).

Cryopreserved, transduced primary T cells were thawed and pre-cultured overnight in OpTmizer medium plus cytokines as described above. The cells were then counted, resuspended in RPMI-10, and added to a V bottom 96 well plate in a 100 μl volume and incubated with (i) a cetuximab biosimilar at the indicated final concentration, (ii) no antibody (0), or (iii) 2,000 ng/ml rituximab biosimilar (R&D Systems) for 15 minutes at 37° C. IL-15 primed NK cells were then added at a 10:1 ratio of NK:CAR-T cells and the V bottom plate was gently centrifuged (100×g, 30 sec) to bring effector and target cells together. After 4 hours of co-culture, remaining CAR+ T cells were identified by FACS. Samples were stained with anti-CD3, anti-CD56, ROR1-Fc, and FVD780, fixed, and acquired on the Ze5 cytometer under volumetric counting mode. Antibody specific ADCC of T cells was assessed by comparing the total live CD56−CD3+ROR1−Fc+ populations treated or not treated with the antibody.

TABLE 5

| Target | Fluorochrome | Antibody | Supplier |
|---|---|---|---|
| EGFR | Alexa 488 | Hu1 (cetuximab biosimilar) | R&D systems |
| EGFR | BV421 | AY13 | BioLegend |
| CD3 | BUV805 | SK7 | Thermo Fisher |
| CD56 | PE | HCD56 | BioLegend |

### Results

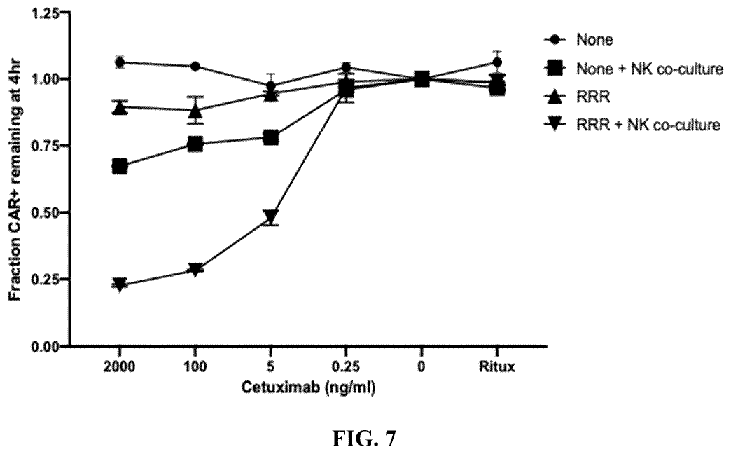

The data show that in bi-cistronic constructs (FIG. 3A), EGFRt lacking a juxtamembrane sequence demonstrated low ADCC in the presence of NK cells and cetuximab, whereas EGFRt with a 3 amino acid RRR juxtamembrane sequence was efficiently killed in the presence of NK cells, even at cetuximab doses as low as 5 ng/ml (FIG. 7).

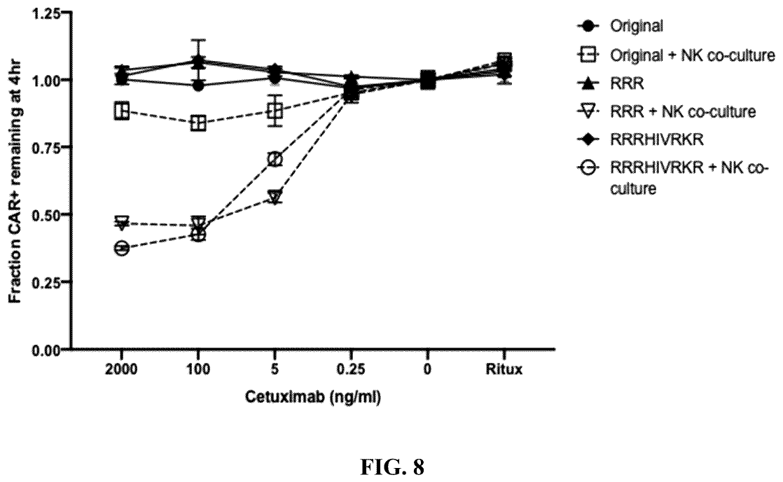

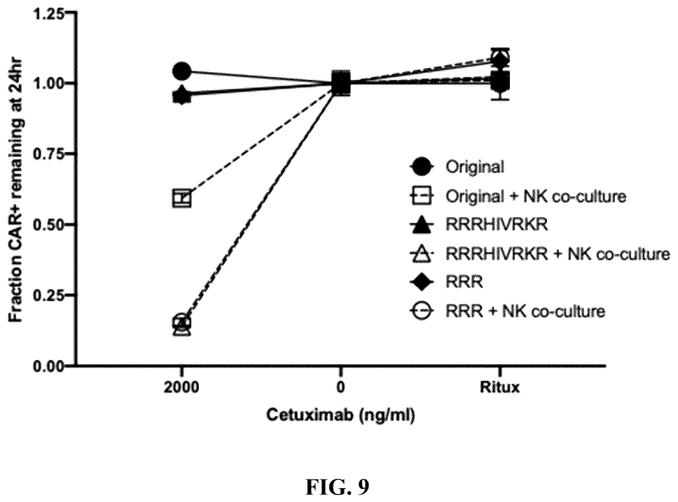

For tri-cistronic constructs with the EGFRt sequence in the 3′ position (FIG. 6A), minimal ADCC was observed for EGFRt lacking a juxtamembrane sequence after 4 hours of treatment. In contrast, EGFRt with RRR or RRRHIVRKR (SEQ ID NO:16) juxtamembrane domains demonstrated significant cetuximab-mediated ADCC in a dose-dependent manner after 4 hours of treatment (FIG. 8). After 24 hours of cetuximab treatment, EGFRt lacking a juxtamembrane sequence exhibited partial ablation in a cetuximab- and NK-dependent manner, whereas EGFRt with RRR or RRRHIVRKR (SEQ ID NO:16) juxtamembrane sequences exhibited nearly complete ablation in a cetuximab- and NK-dependent manner (FIG. 9).

### Example 3: Surface Expression of Additional EGFR-Derived Polypeptides

This example describes studies analyzing the effects of various short and residue-swapped juxtamembrane sequences on the cell surface expression levels of EGFR-derived polypeptides.

To test the minimal sequence requirements for maximizing cell surface expression of EGFR-derived polypeptides, tri-cistronic constructs containing coding sequences for (i) c-Jun, (ii) ROR1-specific CAR, and (iii) an EGFR-derived polypeptide were designed as described in Example 1. These tri-cistronic constructs encoded EGFRt or variants thereof having additional short juxtamembrane sequences: (i) one arginine residue (R), (ii) two arginine residues (RR), (iii) three arginine residues (RRR), or (iv) one arginine residue swapped for lysine (RKR).

Primary T cells from two different human donors were transduced with the indicated tri-cistronic constructs, or were left untransduced. Six days post transduction, cells were stained for ROR1-Fc antigen binding, EGFR expression, and fixable viability dye.

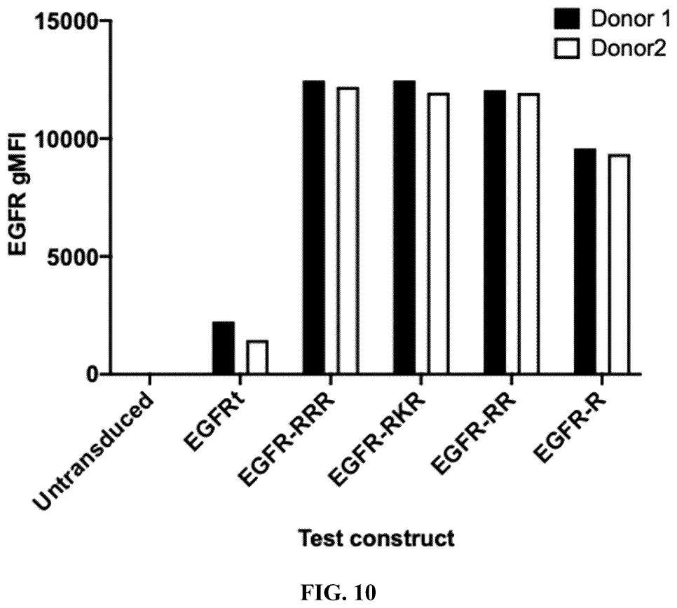

FIG. 10 shows gMFI for anti-EGFR antibody binding in ROR1 CAR+ transduced cells or total live cells in the untransduced condition. As shown in the figure, an EGFR-derived polypeptide containing the RKR juxtamembrane sequence maintained strong cell surface expression. Comparison of cell surface expression levels of EGFR-derived polypeptides containing juxtamembrane sequences with one, two, or three arginine residues (R, RR, or RRR) shows that truncation of the intracellular juxtamembrane domain from RRR to RR reduced surface expression by 2.7%. In contrast, truncation of the intracellular juxtamembrane domain from RRR to R reduced surface expression by 22%.

### Example 4: Kill-Switch Function of EGFR-Derived Polypeptides in Vivo

This example describes studies assessing the effect of the juxtamembrane RRR domain on the in vivo kill-switch function of truncated EGFR following cetuximab administration.

### Methods

### Expression Constructs

The MP71 retroviral vector was used to generate the constructs used in these studies. The vector was modified to incorporate coding sequences for human EGFRt (MP71-EGFRt), a variant having the juxtamembrane domain RRR (MP71-EGFR-RRR), a bi-cistronic CAR expression cassette encoding the mCD19scFv.28z CAR (also annotated as m19.28z or mCD19.28z) and EGFRt or EGFR-RRR (MP71-mCD19scFv.28z.EGFRt/EGFR-RRR; also annotated herein as MP71_m19.28z.P2A.EGFR/EGFR-RRR), or a tri-cistronic CAR expression cassette encoding c-Jun, mCD19scFv.28z, and EGFRt or EGFR-RRR (MP71-cJun.mCD19scFv.28z.EGFRt/EGFR-RRR; also annotated herein as MP71_cJun.T2A.m19.28z.P2A.EGFRt/EGFR-RRR or cJun.m19.28z.EGFRt/EGFR-RRR).

The bi-cistronic CAR constructs included a coding sequence for a CAR (mCD19.28z CAR), which included a murine CD8a signal peptide (UniProt P01731 amino acids 1-27), a murine CD19-specific scFv derived from the ID3 hybridoma (Davila et al., PLoS One (2013) 8(4):e61338), murine CD8a hinge and transmembrane regions (UniProt P01731 amino acids 151-219), a murine CD28 intracellular region (UniProt P31041 amino acids 177-218), and a murine CD3z intracellular domain (UniProt P24161 amino acids 52-164). This CAR-coding sequence was linked by a coding sequence for a P2A self-cleaving peptide sequence to the coding sequence for the human EGFR polypeptide (UniProt P00533 amino acids 334-668 for human EGFRt). For the tri-cistronic construct, a coding sequence for murine c-Jun (UniProt P05627 amino acids 1-334) was cloned upstream of the mCD19.28z CAR coding sequence and linked by a T2A peptide coding sequence.

### Cell Culture, Transduction, and Adoptive Transfer

For retrovirus production, Plat-E cells (Cell Biolabs) were transiently transfected using calcium phosphate (Takara). Supernatants were collected 48 hours later, filtered through 0.45 μm filters, and snap frozen on dry ice prior to storage at −80° C. C57BL/6J and B6.SJL (CD45.1) donor mice were acquired from Jackson Laboratory.

For T cell transductions, single-cell suspensions were obtained from the spleen and peripheral lymph nodes of 6- to 8-week old CD45.1 donor mice and filtered through a 40 μm mesh. Murine CD8⁺ T cells were enriched using negative selection (StemCell) and stimulated with 1 μg/ml plate-bound anti-CD3 (145-2C11) and anti-CD28 (37.51) for 20 hours at 37° C. and 5% CO₂ in complete RPMI (RPMI 1640, 10% heat-inactivated FBS, 1 mM HEPES, 100 U/ml penicillin/streptomycin, 1 mM sodium pyruvate, and 50 μM β-mercaptoethanol) supplemented with 50 U/ml murine IL-2 (PeproTech). Pre-titered retrovirus was loaded onto non-tissue-culture plates pre-coated with 12.5 μg/ml RetroNectin® (Takara) and captured by centrifugation for 2 hours at 2560 rcf at 32° C. Stimulated CD8⁺ T cells were harvested and resuspended at 1×10⁶ cells/ml in complete RPMI supplemented with 50 U/ml IL-2 and anti-CD3/28 mouse T-activator Dynabeads™ (Thermo Fisher) at a 1:1 ratio. Virus-coated wells were aspirated and rinsed with PBS, followed by addition of the T cells, centrifugation at 800 ref for 30 min at 32° C., and incubation at 37° C. in 5% CO₂.

After 24 hours, IL-2-supplemented complete RPMI media was replaced, and T cells were incubated for an additional 24 hours. T cells were harvested, resuspended at 1×10⁶ cells/ml in complete RPMI supplemented with 50 U/ml murine IL-15 (PeproTech), and incubated for an additional 48 hours. Magnetic activator beads were subsequently removed and T cell transduction efficiency (40-60% EGFR⁺) was confirmed by flow cytometry. Transduced cells were then prepared for adoptive transfer by resuspending CD8⁺ T cells at 3×10⁶ EGFR⁺/100 μl in serum-free RPMI 1640 and kept on ice prior to adoptive transfer.

For CAR-T cell adoptive transfer, 6- to 8-week old C57BL/6J mice were pre-conditioned with intraperitoneal injection of 200 mg/kg cyclophosphamide and were injected intravenously by retro-orbital injection with 3×10⁶ EGFR⁺ CAR-T cells after 6 hours. For analysis of peripheral blood, 100 μl blood samples were collected by retro-orbital bleeding into EDTA-coated tubes on the indicated days post CAR-T cell transfer and the blood samples were treated with two rounds of ACK lysis buffer prior to surface staining. Samples were stained using LIVE/DEAD™ Fixable Aqua Dead Cell stain kit (Invitrogen) at 4° C. for 15 minutes. Cells were also stained in the dark at 4° C. for 30 minutes in flow buffer (PBS, 1 mM EDTA, and 2% FBS) with anti-CD8a FITC (53-6.7, BioLegend unless stated otherwise), anti-CD19 PerCP-Cy™ 5.5 (1D3), anti-CD4 PE-Cy™ 7 (RM4-5), anti-CD45.2 APC/Fire™ 750 (104), anti-CD45.1 Brilliant Violet 421™ (A20), hEGFR APC or PE (AY13), and acquired on BD FACSCelesta™ cell analyzer.

For depletion of transferred EGFR⁺ CAR-T cells, cetuximab was infused at 1 mg or 0.1 mg per mouse on day 8. Expansion and depletion of CAR-T cells were monitored in blood samples by flow cytometry. The mice were shown to exhibit B-cell aplasia when the frequency of CD19⁺ B cells was maintained below 3% of the total circulating endogenous CD45.2⁺ cells.

### Results

### EGFR-RRR Exhibits Superior Surface Expression Levels in the Infusion Product

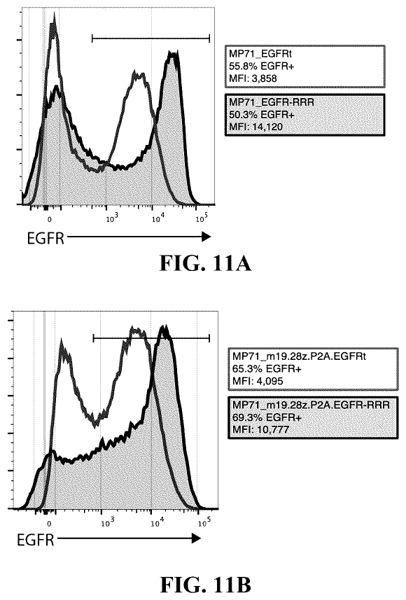

To assess the effect of juxtamembrane modification on the surface expression of truncated EGFR in vitro, murine CD8⁺ T cells were transduced with a retroviral construct comprising the mouse-codon-optimized sequence of human EGFRt with or without an RRR juxtamembrane domain, and analyzed by flow cytometry as described previously. When CD8⁺ T cells were transduced at similar levels (50-56%), EGFR⁺ T cells exhibited >3-fold increase in EGFR-RRR surface expression compared to EGFRt (FIG. 11A).

To determine the effect of the juxtamembrane domain in the context of a multi-cistronic construct, a coding sequence for an EGFR polypeptide was linked by a coding sequence for a P2A self-cleaving peptide to the 3′ end of a coding sequence for a CAR targeting mouse CD19. At similar transduction efficiencies, CAR-T cells transduced with the EGFR-RRR construct displayed high levels of EGFR staining than CAR-T cells transduced with the EGFRt construct (FIG. 11B). Thus, when surface expression is measured by flow cytometry in T cell samples with similar transduction levels, EGFR-RRR exhibited increased surface expression levels on the CAR T cell infusion product.

### EGFR-RRR Is a Stable Target for Antibody-Mediated Depletion in Vivo

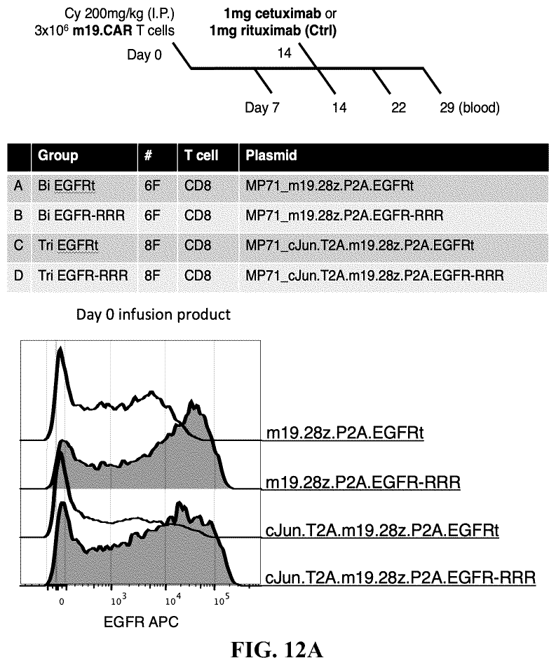

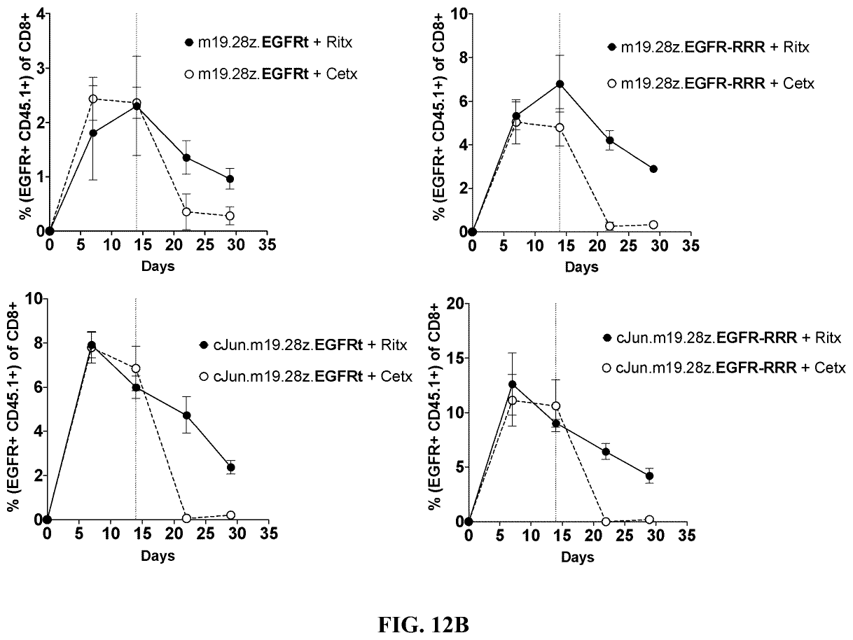

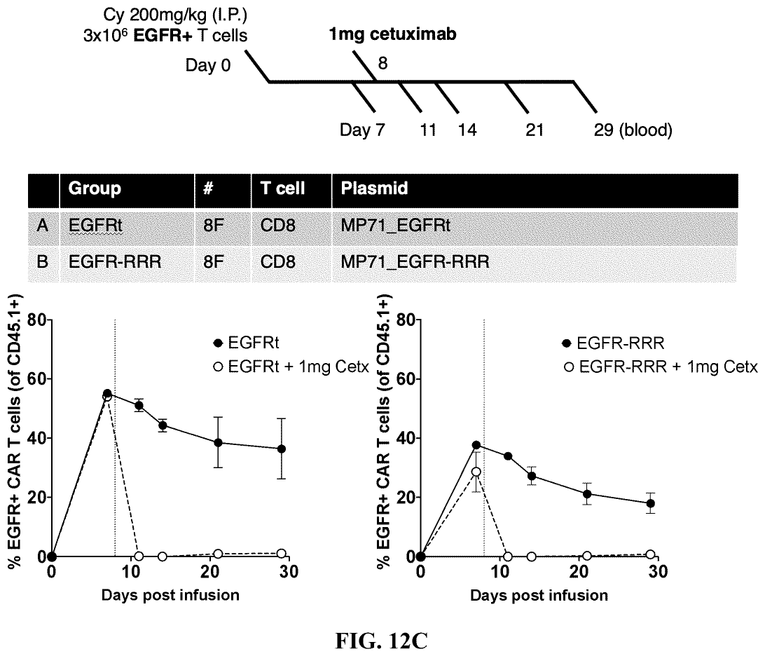

Stably expressed EGFRt can be targeted for depletion with the EGFR-targeting antibody cetuximab (Paszkiewicz et al., J Clin Invest. (2016) 126(11):4262-72). To assess whether EGFR-RRR surface expression is maintained and can be targeted for depletion in vivo, EGFRt or EGFR-RRR were expressed in a bi-cistronic (downstream of mCD19 CAR) or tri-cistronic construct (downstream of cJun.mCD19 CAR) on congenically marked CD45.1⁺ donor CD8⁺ T cells. The data show that EGFR-RRR exhibited higher levels of surface expression in the infusion product (FIG. 12A, bottom). Bulk T cell infusions containing 3×10⁶ EGFR⁺ CAR-T cells were adoptively transferred into lymphodepleted mice. Circulating CAR-T cell levels were tracked in the blood as indicated (FIG. 12A, top). EGFR⁺ CAR-T cells underwent an expansion peak one to two weeks post infusion and declined thereafter.

To confirm that EGFR-RRR can be targeted for depletion, on day 14, half of the mice in each cohort were administered 1 mg of cetuximab or 1 mg of rituximab (control). Following injection, cetuximab depleted a large fraction of EGFRt and EGFR-RRR CAR-T cells, while CAR-T cells were maintained at higher levels in the rituximab cohorts during the observation period (FIG. 12B). Similar depletion results were obtained when T cells expressed EGFRt or EGFR-RRR without CAR (FIG. 12C). These results demonstrate that EGFR-RRR surface expression levels were maintained in vivo and could be efficiently targeted for depletion with cetuximab.

### EGFR-RRR Mediates More Rapid Rebound of B Cells Following T Cell Depletion by Cetuximab

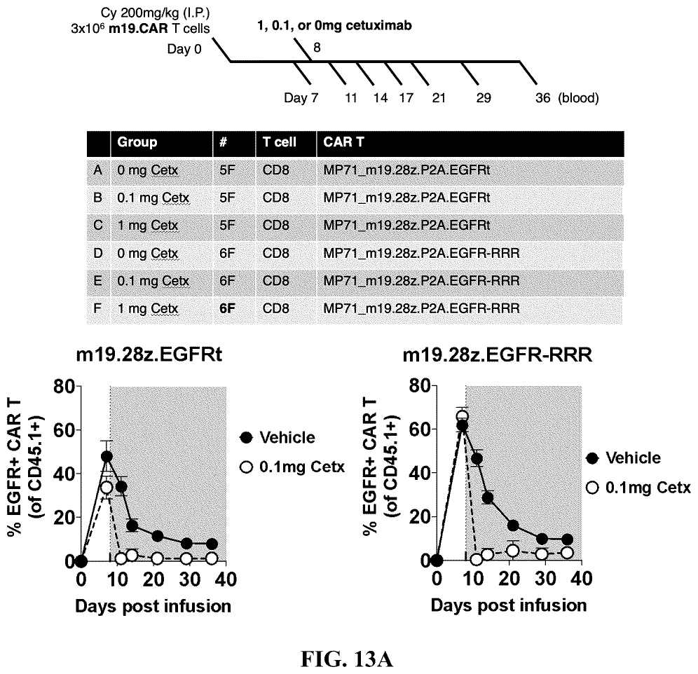

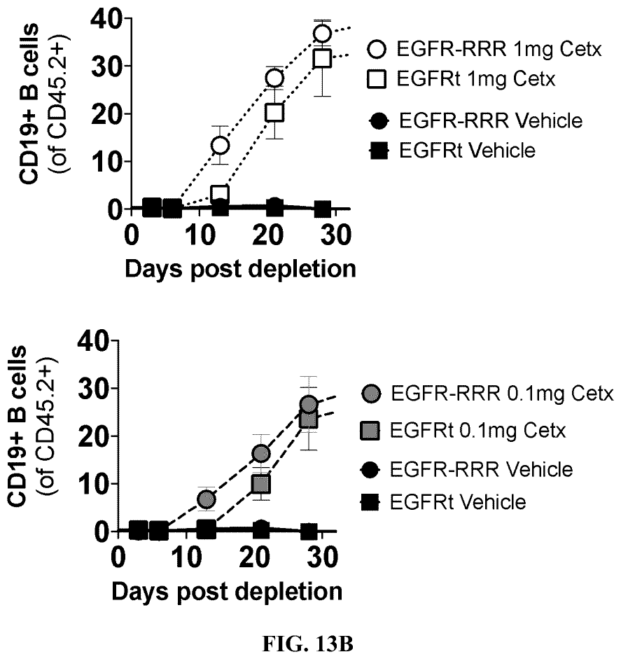

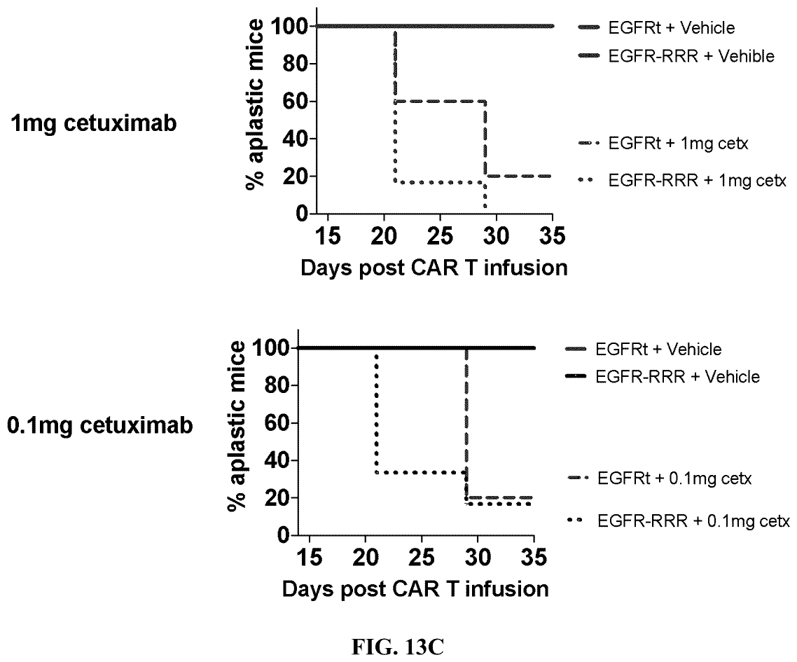

As the addition of the juxtamembrane domain increased EGFR surface levels, we next determined whether EGFR-RRR exhibits differential CAR-T cell depletion kinetics and functional outcomes in vivo. To that end, congenically marked mCD19.28z CAR-T cells expressing EGFRt or EGFR-RRR were adoptively transferred into lymphodepleted mice. T cell engraftment and B cell aplasia were tracked in the blood over time (FIG. 13A). To elucidate differences in the depletion kinetics between EGFRt and EGFR-RRR mCD19.28z CAR T cells, a dose titration of cetuximab was performed. Targeting EGFR for depletion with a single dose of 0.1 mg cetuximab was sufficient to deplete circulating EGFRt and EGFR-RRR CAR-T cells three days following antibody injection. Expression levels of surface EGFR have previously been shown to determine the depletion kinetics of mCD19.28z CAR T cells in vivo (Paszkiewicz et al., 2016). To determine whether EGFR-RRR can mediate more rapid kinetics of B cell rebound post cetuximab, circulating B cells were tracked in mice that received mCD19.28z CAR T cells with or without subsequent depletion. Whereas mice treated with mCD19.28z CAR T cells exhibited sustained B cell aplasia, B cells in cetuximab-treated mice rebounded within 3 weeks of antibody administration. Cetuximab administration in mice previously infused with EGFR-RRR CAR-T cells resulted in more rapid rebound of B cells, including at the lower administered dose (FIG. 13B), and a more rapid resolution of B cell aplasia (FIG. 13C). Therefore, EGFR-RRR mediates more efficient depletion of CAR-T cells, resulting in more rapid shutdown kinetics of the CAR-T cell response following cetuximab.

In summary, mCD19.28z.EGFR-RRR CAR-T cells exhibit higher expression levels of EGFR. This expression was maintained in vivo and was efficiently targeted for depletion with cetuximab. In contrast to EGFRt, targeting EGFR-RRR with cetuximab resulted in complete depletion of mCD19.28z.CAR T cells and more rapid rebound of B cells in vivo.

1. A recombinant polypeptide comprising an extracellular region, a transmembrane region, and an intracellular region, wherein
the extracellular region comprises a human epidermal growth factor receptor (EGFR) Domain III sequence and does not comprise EGFR Domain I or II, and
the intracellular region (i) comprises a juxtamembrane domain that is net-neutral or net-positively charged in the first at least three amino acids (ii) but lacks an active EGFR tyrosine kinase domain.

2. The recombinant polypeptide of claim 1, wherein more than half of the amino acids of the juxtamembrane domain are glycine, serine, arginine, lysine, threonine, asparagine, glutamine, aspartic acid, glutamic acid, tyrosine, tryptophan, histidine, and/or proline.

3. The recombinant polypeptide of claim 1, wherein the amino acid at each position of the juxtamembrane domain is selected according to Table 1.

4. The recombinant polypeptide of claim 1, wherein the juxtamembrane domain comprises RRRHIVRKR (SEQ ID NO:16), RRRHIVRK (SEQ ID NO:17), RRRHIVR (SEQ ID NO:18), RRRHIV (SEQ ID NO:19), RRRHI (SEQ ID NO:20), RRRH (SEQ ID NO:21), RRR, RKR, or RR.

5. The recombinant polypeptide of claim 1, wherein the intracellular region does not contain any residue that is phosphorylated.

6. The recombinant polypeptide of claim 1, wherein the Domain III sequence comprises SEQ ID NO:2.

7. The recombinant polypeptide of claim 1, wherein the extracellular region further comprises, C-terminal to the Domain III sequence, (i) a sequence derived from EGFR Domain IV, (ii) an artificial sequence, or (iii) both (i) and (ii).

8. The recombinant polypeptide of claim 7, wherein the extracellular region comprises amino acids 334-504, 334-525, or 334-645 of SEQ ID NO:1.

9. The recombinant polypeptide of claim 1, wherein the transmembrane region is derived from a human EGFR transmembrane domain.

10. The recombinant polypeptide of claim 1, further comprising a signal peptide derived from human EGFR, human granulocyte-macrophage colony-stimulating factor (GM-CSF), human Ig kappa, mouse Ig kappa, or human CD33.

11. The recombinant polypeptide of claim 1, comprising SEQ ID NO:26, 27, 28, 29, 30, 31, 32, 33, 34, 35, 36, 37, 38, 39, or 40; or an amino acid sequence at least 90% identical thereto.

12-18. (canceled)

19. A cell expressing the recombinant polypeptide of claim 1.

20. The cell of claim 19, wherein the cell is a human T cell.

21. A pharmaceutical composition comprising the cell of claim 19 and a pharmaceutically acceptable carrier.

22. A method of treating a patient in need thereof, comprising administering the cell of claim 19 to the patient.

23. The method of claim 22, wherein the patient has cancer and the cell is a T cell expressing a CAR, a T cell receptor (TCR), an engineered TCR, or a TCR mimic that is specific for a tumor antigen present in said cancer.

24. The method of claim 22, further comprising administering to the patient an effective amount of an antibody specific for human EGFR once the patient has been treated, wherein the antibody elicits cytotoxicity against cells expressing the recombinant polypeptide, and optionally the antibody is IgG1 or IgG2.

25-26. (canceled)

27. The recombinant polypeptide of claim 9, wherein the transmembrane region comprises SEQ ID NO:5.

28. The method of claim 22, wherein the cell is derived from the patient.

29. The method of claim 24, wherein the antibody is cetuximab.
